(19) United States
(12) Patent Application Publication
(10) Pub. No.: US 2024/0360272 A1
(43) Pub. Date: Oct. 31, 2024

(54) TEXTILES DERIVED FROM FOOD WASTE AND PROCESSES OF MAKING THEREOF

(71) Applicant: ALT Tex Inc., Brampton (CA)

(72) Inventors: Avneet Ghotra, Brampton (CA); Zubia Arshad, Brampton (CA); Samantha Cheung, Brampton (CA)

(73) Assignee: ALT Tex Inc., Brampton, ON (CA)

(21) Appl. No.: 18/682,735

(22) PCT Filed: Aug. 17, 2022
(86) PCT No.: PCT/IB2022/057716

§ 371 Date: Feb. 9, 2024

Related U.S. Application Data
(60) Provisional application No. 63/234,471, filed on Aug. 18, 2021.

(51) Int. Cl. C08G 63/08; C08G 63/78; C08K 3/22; C08K 5/29; C08K 5/526; C08K 13/02; C08L 67/04; C12N 1/14; C12N 9/24; C12R 1/845; D01F 1/10; D01F 6/62; D01F 6/94

(52) U.S. Cl. CPC C08G 63/08; C08G 63/78; C08K 13/02; C08L 67/04; C12N 1/145; C12N 9/2402; D01F 1/10; D01F 6/625; D01F 6/94; C08G 2230/00; C08K 2003/2241; C08K 5/29; C08K 5/526; C08K 2201/014; C08L 2201/06; C08L 2201/08; C08L 2203/12; C08L 2205/06; C12R 2001/845; D10B 2401/063; D10B 2401/12; D10B 2501/00

(57) ABSTRACT

The present disclosure is directed to methods of converting food waste to polymers comprising poly(lactic) acid and an additive and biodegradable products thereof. The methods include providing a feedstock comprising food waste, hydrolyzing the feedstock with an enzyme and fermenting the feedstock with a microbe to produce a mixture, isolating lactic acid from the mixture, converting the lactic acid to poly(lactic) acid, adding, reacting, grafting, and/or cross-linking an additive to the poly(lactic) acid to produce a polymer mixture, and forming the polymer with the polymer mixture.

### CROSS-REFERENCE TO RELATED APPLICATIONS

This application claims the benefit of priority to U.S. Provisional Patent Application No. 63/234,471, filed Aug. 18, 2021, which is hereby incorporated by reference in its entirety for any and all purposes.

### BACKGROUND

Textiles are fiber-based materials that are interlaced by a variety of methods, such as weaving, knitting, webbing or felting. The fiber-based materials can include natural fibers (e.g., plant-derived fibers (e.g., cotton or linen), or animal derived fibers (e.g., wool or silk)), man-made fibers (e.g., polyester and rayon), threads, yarns, or filaments. To create a textile, the fibers are extruded, spun and then woven, knitted, webbed or felted into a fabric.

### SUMMARY

The present disclosure is directed to methods of converting food waste to polymers comprising poly(lactic) acid and an additive and products thereof. The poly(lactic) acid of the present disclosure is biodegradable, and may be extruded and/or formed into filaments and/or fibers (for use in a yarn and/or thread) for textile manufacturing, or for the manufacture of single use plastic alternatives.

In one aspect, the present disclosure provides a method for producing a polymer, the method comprising: providing a feedstock, where the feedstock comprises food waste; hydrolyzing the feedstock with an enzyme and fermenting the feedstock with a microbe to produce a mixture; isolating lactic acid from the mixture; converting the lactic acid to poly(lactic) acid; adding, reacting, grafting, and/or cross-linking an additive to the poly(lactic) acid to produce a polymer mixture; and forming the polymer with the polymer mixture; wherein the polymer is biodegradable. The polymer mixture may be a mixture of the poly(lactic) acid with one or more additives in the mixture, grafted to the poly(lactic) acid, and/or cross-linking the poly(lactic) acid or another polymer.

The enzyme may include cellulase and/or microbe may include Rhizopus oryzae.

In another aspect, the present disclosure provides a method for producing a polymer comprising poly(lactic) acid, the method comprising: providing a feedstock, where the feedstock comprises food waste; hydrolyzing the feedstock with an enzyme, where the enzyme comprises cellulase and fermenting the feedstock with a microbe, where the microbe comprises Rhizopus oryzae, to produce a mixture; isolating lactic acid from the mixture; converting the lactic acid to poly(lactic) acid; adding, reacting, grafting, and/or cross-linking an additive to the poly(lactic) acid to produce a polymer mixture; and/or forming the polymer with the poly(lactic) acid or the polymer mixture; wherein the polymer is biodegradable.

In another aspect, the present disclosure provides a plastic alternative, a single use plastic alternative, a fiber, filament, yarn, textile or fabric made by the method disclosed herein.

In a related aspect, the present disclosure provides a polymer comprising poly(lactic) acid and an additive. The polymer is biodegradable and exhibits a tensile strength of about 30 MPa to about 90 MPa, a Young's modulus of about 500 MPa to about 2700 MPa, or a combination thereof.

In another aspect, the present disclosure provides a plastic alternative, a single use plastic alternative, a fiber, a filament, a yarn, a textile or a fabric comprising the polymer described herein.

In another aspect, the present disclosure provides a method of producing a lactic acid, the method comprising: providing a feedstock comprising food waste selected from the group consisting of starch-based food waste, fructose-based food waste, cellulose-based food waste, sucrose-based food waste, or combinations of two or more thereof; contacting the feedstock with an enzyme blend comprising one or more enzymes; fermenting the feedstock with an immobilized fungal strain to produce a fermentation mixture; and isolating lactic acid from the fermentation mixture. In some embodiments, the contacting step comprises grinding, sterilizing, and/or hydrolyzing the feedstock.

In yet another aspect, the present disclosure provides a method of producing a biodegradable polymer comprising poly(lactic acid), the method comprising: providing a lactic acid; converting the lactic acid to oligo(lactic acid); depolymerizing the oligo(lactic acid) to produce cyclic lactide with a catalyst selected from the group consisting of Zn(La)₂, sodium bicarbonate, Sn(OEt)₂, ZnO, TnO₂, Zn acetate, or combinations of two or more thereof; polymerizing the cyclic lactide to produce the poly(lactic acid); and contacting or combining the poly(lactic acid) with an additive to produce a polymer mixture.

In another aspect, the present disclosure provides a method of producing a biodegradable filament, the method comprising: providing a polymer made by the method described herein; and extruding or pelletizing the polymer into multifilament and/or monofilament fibers.

In another aspect, the present disclosure provides a biodegradable polymer comprising poly(lactic acid) made by the method described herein.

The polymer may be in the form of a plastic alternative (or may be a suitable for a single use plastic alternative), or a fiber and/or filament (suitable for use in a yarn and/or thread), fastener, and/or accessory suitable for use in textile manufacturing, or combinations of two or more thereof.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings, FIGS. 1-7 show flowcharts illustrating the process for converting post-industrial food waste, into lactic acid via hydrolysis and fungal fermentation, followed by purification and polymerization into poly(lactic) acid, melt-blending/reaction with additives to create a textile-specific PLA, and production of biodegradable PLA textiles.

FIG. 1 depicts an illustrative method of producing an L-lactic solution from post-industrial food waste containing large polysaccharides, in accordance with present implementations.

FIG. 2 depicts an illustrative method of producing an oligomeric poly-L-lactic acid from the L-lactic solution of FIG. 1, in accordance with present implementations.

FIG. 3 depicts an illustrative method of producing an L-lactide isomer from the oligomeric poly-L-lactic acid of FIG. 2, in accordance with present implementations.

FIG. 4 depicts an illustrative method of producing a recrystallized L-lactide from the L-lactide isomer of FIG. 3, in accordance with present implementations.

FIG. 5 depicts an illustrative method of producing a poly-lactic acid polymer from the recrystallized L-lactide of FIG. 4, in accordance with present implementations.

FIG. 6 depicts an illustrative method of producing a purified poly-lactic acid polymer from the poly-lactic acid polymer of FIG. 5, in accordance with present implementations.

FIG. 7 depicts a schematic process for converting post-industrial food waste to biodegradable polymers (i.e., fiber) and textile using the methods disclosed in the present disclosure.

### DETAILED DESCRIPTION

Lactic acid can be produced by chemical synthesis or biological production (e.g., microbial fermentation). The chemical production of lactic acid relies on the hydrolysis of lactonitrile derived from acetaldehyde and hydrogen cyanide. Chemical synthesis is technically challenging and it generally yields a mixture of two optical isomers, d-(−)- and l-(+)-lactic acids. Biological synthesis is carried out by bacterial fermentation of simple sugars using bacterial species, such Lactobacillus and Lactococcus. Biological production has several advantages over chemical synthesis. First, it can yield one form of lactic acid isomer alone, or a mixture both isomers in different proportion, depending on the microorganism, substrate and growth conditions used. Second biological production is cheap because the raw materials (e.g., whey, molasses, starch, beet, sugar cane and other carbohydrate-rich materials) is widely available. Sugars and starches can be used as substrates for biological production of lactic acid. Starch can be a plentiful, inexpensive and renewable material that can be found in a large variety of plant sources, such as grains, tubers, and fruits.

Due to widespread environmental concerns, there has been significant pressure on companies to discontinue the use of plastic in favor of more environmentally safe materials. Lactic acid can be used as a building block in the manufacture of biodegradable polymers. One of the most promising application of lactic acid is its use for biodegradable and biocompatible polylactate polymers, such as poly-lactic acid (PLA), which are considered to be environmentally friendly alternatives to petroleum-based plastics.

Despite the commercial and environmental advantages of poly(lactic) acid use in various applications (e.g. single use cutlery, etc), there remains a need in the art for apparel-grade fabric (e.g., textile) made with poly(lactic) acid polymers. Additionally, there is a need for efficient, low cost processes and compositions for producing poly-lactic acid polymers for use in textile manufacturing. The present disclosure addresses these needs.

### Method for Producing a Polymer

In the United States, about 30-40% of the food supply is wasted annually, which has an approximate value of $160 billion. The present disclosure provides methods for repurposing food waste into biodegradable textiles (e.g., woven, non-woven, or knit), fabrics, fibers, filaments, fasteners, and/or accessories, suitable for use in textile manufacturing.

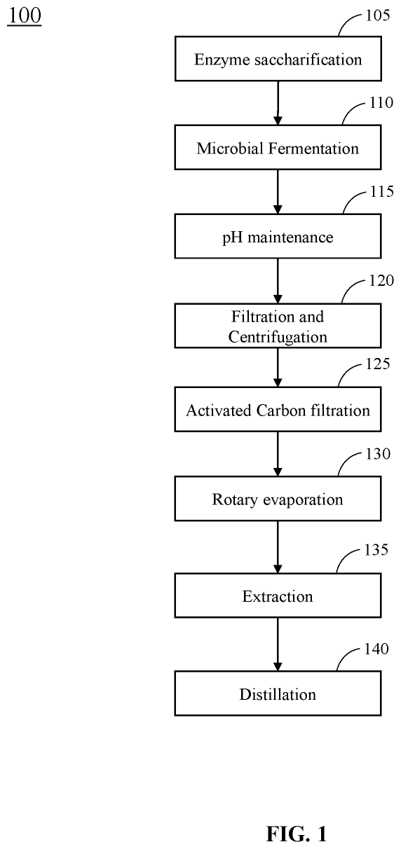

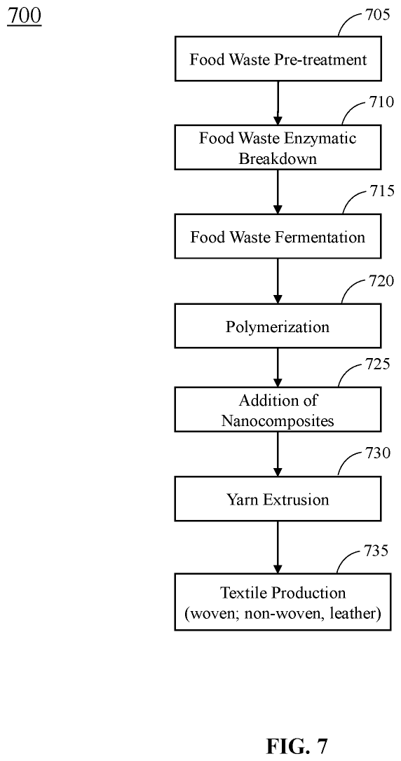

The methods disclosed herein provide an exclusive use of post-industrial food waste, processed through immobilized fungal fermentation technology to create sterically pure L-lactic acid with minimal byproducts, which is then purified and used as input for polylactic acid (PLA) production for textiles. The PLA is then extruded with performance-enhancing additives into apparel-specific textiles (e.g., fabrics). The methods disclosed herein comprise three main steps. The process starts with the conversion of food waste to Lactic acid (FIG. 1), the lactic acid is then converted to poly-L-lactic acid (PLLA) (FIGS. 1-6), which is converted to apparel-grade filaments (FIG. 7). The method disclosed herein produced a polymer that showed synergistic and emergent properties, such as improved tensile strength, elongation/flexibility, reduced hydrolysis and boiling shrinkage, improved thermal stability, and softer feel of the fabric. For example, the elongation at break is about 112.7% to about 130.73% depending on the blend of additives used when compared to a commercial polymer with 8% elongation at break point (Table 1).

The production of biobased or biodegradable polymers from food and/or food waste may be known in the art. However, the methods used in the prior art rely on materials such as recycled polyester to produce fabrics, orange peels to make leather, and/or food waste to make other types of polymers such as PHA, PBS (partially), etc. The PLA polymer production methods used in the art utilize food crops such as beet, sugar cane, corn/maize, cassava for their polymer production.

The present disclosure is the first to demonstrate the creation of an apparel (i.e., textile) specific PLA-fabric (e.g. textile) by utilizing post-industrial food waste for its input to convert food waste to lactic acid. The creation of apparel-grade PLA from food waste is made possible by an innovative combination of fungal fermentation (e.g., immobilized fungal fermentation) and a unique blend of performance-enhancing additives.

### Pre-Fermentation

The present disclosure also provides innovative pre-fermentation and fermentation processes that are not known in the art. For instance, the food waste can undergo pre-fermentation process may comprise the steps of grinding, sterilizing, steam explosion, and hydrolyzing the input food waste. In some cases, the pre-fermentation process may comprise the steps of grinding and sterilizing, but no hydrolysis. The hydrolysis step may be omitted when the waste input contains a high concentration of short saccharides (i.e monosaccharides, disaccharides). The pre-fermentation process may comprise the hydrolysis of complex polysaccharides to short saccharides (i.e monosaccharides, disaccharides).

A novel blend of enzymes may be used to hydrolyze the complex polysaccharides. The novel blends of enzymes disclosed herein is catered and customized for each type of food waste input. Alternatively, a novel blend of enzymes disclosed herein may be optimized for various food waste groups/types. The hydrolysis with the novel blend of enzymes may take place a temperature of about 15° C. to about 70° C., optionally at about 50° C.; and pH of about 3 to about 6, optionally at about 5.5. For example, for potato waste hydrolysis, the enzyme blend may include about 1% glucoamylase, about 1% a-amylase, about 0.4% arabanase, about 0.4% cellulase, about 0.4% beta-glucanase, about 0.4% hemicellulase, and about 0.4% xylanase at a temperature of about 50° C. and pH of about 5.5.

### Fermentation

The fermentation process uses a combination of unique strains of microorganisms. For example, a Rhizopus NRRL 295, or a variant thereof may be used. In some cases, Rhizopus oryzae NRRL295 (the original strain) may be used for most food waste feedstocks. Alternatively, engineered microbial strains (e.g, adaptive evolution strain) may be used for few feedstock hydrolysis. The microorganisms disclosed herein are resistant to stress (e.g., can grow in the presence of complex polysaccharides), lower pH (acidic pH such as 4-5), high temperatures (40° C.-45° C.). In addition, the microorganisms disclosed herein produced high yield of lactic acid when grown in stressful conditions when compared to microorganisms used in the art. Example of generic microbes used for lactic acid fermentation may include Lactobacillus rhamnosus, Lactobacillus manihotivorans, Lactobacillus plantarum, Lactobacillus delbrueckii.

The present disclosure is also the first to demonstrate that immobilized forms of NRRL 395 Rhizopus oryzae in a lactic acid fermentation modulated by MgO or Mg(OH)₂ pH control, enhanced the yield of L-lactic acid by about 70% when compared to control stains (e.g., unimmobilized Rhizopus). As a fungal species, Rhizopus oryzae, is specific for L-Lactic acid and produces minimal levels of byproducts. The Rhizopus species disclosed herein was optimized by the present inventors for L-lactic acid yields as high as 140 g/L. In contrast, the prior art discloses L-lactic acid yields that are much lower (e.g, as high as 100 g/L) for semi-continuous or continuous fermentations. See e.g., Pimtong, V. et al., Process Biochemistry, 52: 44-52 (2017); Lin, J. et al., Chemical Engineering and Processing: Process Intensification, 46(5): 369-374 (2007). The optimized performance of the Rhizopus species disclosed herein may be achieved through procedures involving: (1) adaptive evolution for over 3 years (e.g., genetic modification techniques such as random mutagenesis cycles, and/or shotgun mutagenesis based tools); (2) self-immobilization method (e.g. pellet formation); and/or (3) physical and/or scaffold immobilization methods (on a scaffold of various shapes and materials). These combinations of methods generated optimized Rhizopus strain(s) (e.g., more enhanced Rhizopus strain when compared to wild-type strains and/or strains used in the art) for lactic acid (LA) production with optimized conditions.

The present disclosure demonstrates that immobilization of the fungal strain during fermentation was a key factor in increasing yield of L-lactic acid produced from waste food. Indeed, the immobilized (e.g., optimized) Rhizopus strain produced L-lactic acid yields as high as 140 g/L while the lactic acid yield from the original (not immobilized) Rhizopus strain was about 40 g/L. The genetic modification of the Rhizopus strain (e.g., genetically modified and/or recombinant Rhizopus strain) when combined with immobilization further enhanced the yield of L-lactic acid produced from the food waste. Surprisingly, the genetically modified Rhizopus strain also reduced the amount of byproduct generated during fermentation. For example, the production of bioproducts, such as ethanol, fumaric acid, and/or acetic acid was minimized when compared. Specifically, the fumaric acid production during fermentation was reduced to 0.0 g/L (e.g., by HPLC analysis) compared to 0.22 g/L to 1.36 g/L; ethanol production was reduced to less than 0.6 g/L to compared to 3 to 4 g/L and acetic acid production was reduced to less than 0.01 g/L compared to 1 to 2 g/L.

The present disclosure also discloses for the first time that using magnesium oxide (MgO) or magnesium hydroxide Mg(OH)₂ as the alkaline source for pH adjustment also enhanced the yield of L-lactic acid generated from food waste. MgO and Mg(OH)₂ are not commonly used as alkaline sources for pH adjustment in the art. The most common alkaline sources are sodium hydroxides (NaOH), calcium carbonate (CaCO₃), CaOH₂, etc. The inventors of the present disclosure substituted CaCO₃ with Mg(OH)₂ or MgO because of their eco-friendly procedure and non-toxic byproducts. It is well established that the industry standard alkaline source for pH adjustment is CaCO₃. However, CaCO₃ generates significant amounts of toxic byproduct, such as calcium sulphate (also known as gypsum). As such, the observation that substituting Mg(OH)₂ or MgO enhanced L-lactic acid yield was surprising and unexpected, when combined with the immobilized microbial fermentation which has not been attempted before. Indeed, the addition of Mg(OH)₂ or MgO enhanced the survival and growth of immobilized Rhizopus strains. Thus, Mg(OH)₂ or MgO allowed self immobilization (e.g., Rhizopus strains) to survive.

### Lactic Acid Polymerization

The lactic acid generated from the food waste may be purified using techniques known in the art. For example, the L-lactic may be purified via centrifugation, activated carbon use, filtration, rotary evaporation, and ionic exchange chromatography or liquid-liquid extraction. This may be the first time ionic exchange chromatography has been used at scale in the industry to purify L-lactic acid (L-LA). Scaled weak and/or strong anion and cation exchange resins made of materials such as acrylic, polymer, styrene-divinylbenzene (gel), styrene-divinylbenzene (macroreticular), hyper-crosslinked polystyrene divinylbenzene, polystyrene, gel, crosslinked acrylic acid copolymer, etc may be used in the methods of the present disclosure. Solvents such as HCl, NaOH, methanol for elution and/or resin regeneration may also be used (e.g., concentrations of 0.1M to 2M). For example, gel-based strong acid cation exchange resin may be used to convert dissociated lactic acid in fermentation broth into undissociated form, followed by acrylic-gel weak base anion exchange resin chromatography to separate lactic acid from the fermentation impurities at flow rates of 12 Bed volumes/hour with L-LA:resin ratio of 1:2 with use of 1 M HCl for elution.

The purified L-lactic acid is then converted to Lactide via dehydration, oligomerization, and depolymerization using customized and optimized reaction conditions (e.g., temperature, pressure, atmospheric conditions). As with the enzymes disclosed herein, the present disclosure also provides novel blend of catalysts selected from (e.g., Zn(La)₂, sodium bicarbonate, Ca(OMe)₂, (C₉H₂₁O₃)Y, Al(OiPr)₃, Sn(OEt)₂, ZnO (or various particle sizes), TnO, Zn acetate, creatinine, SnO, Al₂SzO₃, SiO, SnCl₂, Sb₂O₃, SnHPO₃, Tin powder, tin alkoxides, tin halides, SnO₂, Pb oxide, beta zeolites, sulfuric acid, SnIPO₃, CsCO₃, any ZIF systems, or combinations of two or more thereof. In any embodiment, the monomer to catalyst ratio is between about 0.0001% to about 10% optionally, about 0.75% of the catalyst. For example, the catalyst blend may include 0.5% Zn(La)2 and 0.2% sodium bicarbonate. Alternatively, the catalyst blend may include 0.6% ZnO and 0.4% sodium bicarbonate.

The purified lactic acid may then be converted to lactide, which may further be converted to PLA using e.g., a ring-opening polymerization. The ring-opening polymerization may also comprise one or more catalyst disclosed herein.

In any embodiment, the polymerizing the cyclic lactide to produce the poly(lactic) acid may include heating. The heating may be at a temperature above about 100° C. The temperature is between about 100° C. to about 300° C., about 110° C. to about 250° C., about 120° C. to about 220° C., about 140° C. to about 220° C., or about 160° C. to about 210° C. The heating may be conducted under an inert gas (e.g., N₂ or argon). The heating may occur for about 5 minutes to about 10 hours or more, about 10 minutes to about 5 hours, or 20 minutes to about 4 hour. The polymerizing may occur with or without solvents, such as toluene, which can be replenished throughout the reactions. In any embodiment, the polymerizing may further include adding a catalyst. The catalyst may include Ca(OMe)₂, (C₉H₂₁O₃)Y, Al(OiPr)₃, Sn(Oct)₂, ZnO, Zn acetate, Zn(lactate), aluminum isopropoxide, lanthanum isopropoxide, tin isopropoxide, Guanidinate Zn(II) complex, Zinc complexes with any R groups, Calcium complexes with any R group ligands. Barium complexes with any R group ligands, strontium complexes with any R groups, tin complexes with any R group ligands, germanium complexes with any R group ligands, copper complexes with any R group ligands, Zirconium complexes with any R group ligands, titanium complexes with any R group ligands, aluminum triisopropoxide, salen metal alkoxides, DBU, bismuth subsalicylate, bismuth (III) acetate, bismuth methylene, Bismuth lactate, bismuth complexes (with any R groups), Iron subsalicylate, Aluminum subsalicylate, Iron octoate, copper octoate, zinc octoate, zinc lactate, iron chlorides, creatinine glycolate/acetate, thiozole carbenes, imidozolium carbenes, imidazolium carbenes, pyridine-derived bases (e.g. DMAP (4-(dimethylamino)-pyridine), DMAP-DCC, DMAP-MSA, PPY (4-(dimethylamino)-pyridine)), guanidine and amidine derivatives (e.g. 1,5,7-tri-azabicyclododecene (TBD), N-methyl-1,5,7-triazabicyclododecene (MTBD), di-azabicycloundecene (DBU), N-heterocyclic carbenes, phosphazenes, B-ketoiminate, Zn phenoxide, Fe(III) alkoxide, Di-zinc complex, addition of initiator if necessary (1-dodecanol, thiol urea, pentadiol, triethylene glycol. EG, PEG variaties, PGA, pyrenebutanol, amines, alcohols of varying lengths and functional groups (diols. triols, tetra-ols)), TiO, or combinations of two or more thereof.

In any embodiment, the monomer to catalyst ratio may be from about 10:1 to about 10,000:1, about 100:1 to about 6000:1, about 400:1 to about 4000:1, about 600:1 to about 2000:1, or about 800:1 to about 1500:1. For example, polymerization may be conducted at a temperature of about 140° C., under nitrogen gas, with monomer to catalyst ratio (M:C ratio) of about 1000:1 with Guanidinate Zn(II) complex. In another example, polymerization may be conducted at 160° C., under nitrogen gas, with M:C ratio of about 1000:1 with Sn(EtO2)/Tin octanoate.

In any embodiment, the method may further include purifying the poly(lactic) acid. The purifying may include dissolving and precipitating the poly(lactic) acid followed by filtering. The dissolving and precipitating may be conducted with an organic solvent (e.g., C1-C5 alcohol such as chloroform or methanol). The purification process may involve dissolving polymer in chloroform completely at varying temperatures and precipitating using methanol. The purification may involve rapid purification cycles involving submerging, partially dissolving, and stirring the crude polymers in combined mixtures of chloroform:methanol at ratios of about 5:1 to about 1:5. In any embodiment, the purification may involve submerging or partially dissolving the crude polymers in a chloroform:methanol mixture ratio of about 1:4 and heating the mixture at about 50° C. to about 70° C. for about 15 minutes to about 180 minutes, optionally for about 90 minutes. In any embodiment, the purification can occur at temperatures from about 0° C. to about 100° C. for 5 minutes to 6 hours with or without stirring/agitation.

### Apparel-Grade Filaments

The apparel-grade filament (i.e., the polymer) of the present disclosure is generated using a special combination of elements that act synergistically to produce unexpectedly high quality apparel-grade filaments with improved tensile strength, elongation/flexibility, reduced hydrolysis and boiling shrinkage, improved thermal stability, and/or softer feel of the fabric compared to the commercially available PLA-derived polymer (Table 1). In particular, the process disclosed herein combines tris(nonylphenyl) phosphite (TNPP), chain extenders (e.g., multi-functional epoxy-based compatibilizer), nucleating agents (e.g., TiO2), anti-hydrolysis agents (e.g., carbodiimides (epoxy or non-epoxy version)), elasticity agents (e.g., tensile modulus; (e.g., polyether block amide, TPU)), and tensile strength agents (e.g., polyether block amides, polyamides). For example, the apparel-grade filament was produced by reacting and/or blending the poly-lactic acid polymer with 10% polyether block amide, 0.15% TNPP, 1% TiO2, and 1% epoxy-based chain extenders. In some embodiments, the apparel-grade filament comprises poly-lactic acid polymer, polyether block amide, TNPP, TiO2, epoxy-based chain extenders, and/or any combination of additives disclosed herein.

Accordingly, one aspect of the present provides a method for producing a polymer (e.g. biodegradable polymer) comprising: providing a feedstock (e.g., the feedstock comprises food waste); hydrolyzing the feedstock with an enzyme and fermenting the feedstock with a microbe to produce a mixture; isolating lactic acid from the mixture; converting the lactic acid to poly(lactic) acid; adding, reacting, grafting, and/or cross-linking an additive to the poly(lactic) acid to produce a polymer mixture; and forming the polymer with the polymer mixture.

Another aspect of the present disclosure provides a method for producing a polymer comprising poly(lactic) acid, the method comprising: providing a feedstock (e.g., the feedstock comprises food waste); hydrolyzing the feedstock with an enzyme and fermenting the feedstock with a microbe to produce a mixture; isolating lactic acid from the mixture; and converting the lactic acid to poly(lactic) acid. In some embodiments, the method optionally comprises: adding, reacting, grafting, and/or cross-linking an additive to the poly(lactic) acid to produce a polymer mixture; and/or forming the polymer with the poly(lactic) acid or the polymer mixture. In some embodiments, the enzyme comprises cellulase and the microbe comprises Rhizopus oryzae. In some embodiments, the polymer is biodegradable.

Another aspect of the present disclosure provides a polymer comprising poly(lactic) acid and an additive. In some embodiments, the polymer is biodegradable and exhibits a tensile strength of about 30 MPa to about 90 MPa, a Young modulus of about 500 MPa to about 2700 MPa, a molecular weight (MN) of about 90,000 to about 160,000, elongation of 5% to 400%, and improvement in glass temperature and thermal decomposition temperature (thermal stability) by 10% to 100%.

### MODES FOR CARRYING OUT THE DISCLOSURE

FIG. 1 depicts an illustrative method of producing an L-lactic solution from post-industrial food waste containing polysaccharides of various sizes, in accordance with present implementations. The method 100 can include providing an enzyme, such as e.g., 3% cellulose, to initiate the saccharification of the food waste containing large polysaccharide chains (ACT 105). The method 100 can include initiating the fermentation process by adding a microbe, such as e.g., Rhizopus oryzae. (ACT 110). In the method, ACT 105 and ACT 110 can occur simultaneously. The method 100 can include adjusting the pH of the fermentation mixture by e.g., adding an alkaline source such as e.g., CaCO₃, MgO, or Mg(OH)₂ (ACT 115). The method 100 can include reaction conditions, such as e.g., 50 rpm/T=40° C./pH=6. The method 100 can include filtrating the fermentation mixture followed by centrifugation for about e.g., 30 minutes (ACT 120). Impurities may then be removed from the lactic acid using activated carbon (ACT 125). The purified lactic acid is the concentrated via e.g., rotary evaporation (e.g., a T=60° C.) (ACT 130). The L-lactic acid is then extracted using e.g., sonication with e.g., ethyl acetate (ACT 135); and isolated using e.g., distillation (e.g., T=40° C.) (ACT 140).

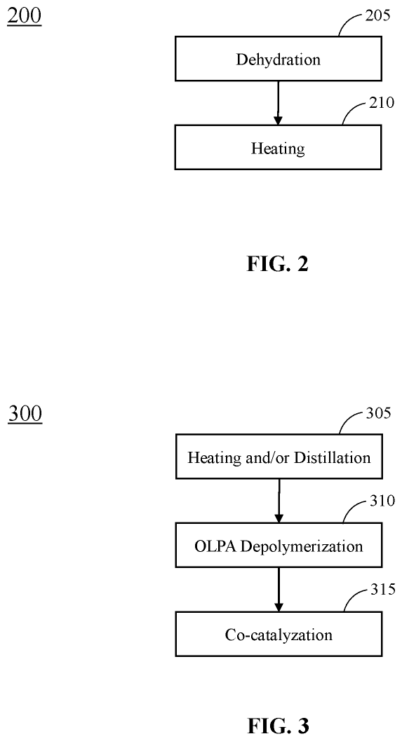

FIG. 2 depicts an illustrative method of producing an oligomeric poly-L-lactic acid from the L-lactic solution of the method 100, in accordance with present implementations. The method 200 can include supplying and/or adding air or an inert gas (e.g., N₂) to the L-lactic acid solution with pressure reduction (e.g., 4000 Pa) (ACT 205); and then heating the solution a high temperature (e.g., T=150° C.) for a specified time (e.g., 5 hours) (ACT 210) to generate an oligomeric poly-L-lactic acid (OLPA).

FIG. 3 depicts an illustrative method of producing an L-lactide isomer from the oligomeric poly-L-lactic acid of the method 200, in accordance with present implementations. The method 300 can include heating the reaction and/or using rectification columns as pre-step (to e.g., T=160° C.) (ACT 305). Then, adding e.g., Zn(La)₂ to the reaction and depolymerizing the OLPA via e.g., vacuum distillation. (ACT 310). The method 300 can include co-catalyzing the reaction mixture with e.g., NaHCO₂ for a specified time (e.g, 2.0 hrs). (ACT 315) to generate an L-lactide isomer. The method 300 can include reaction conditions, such as T=195° C./P<400 Pa/3.25 hrs.

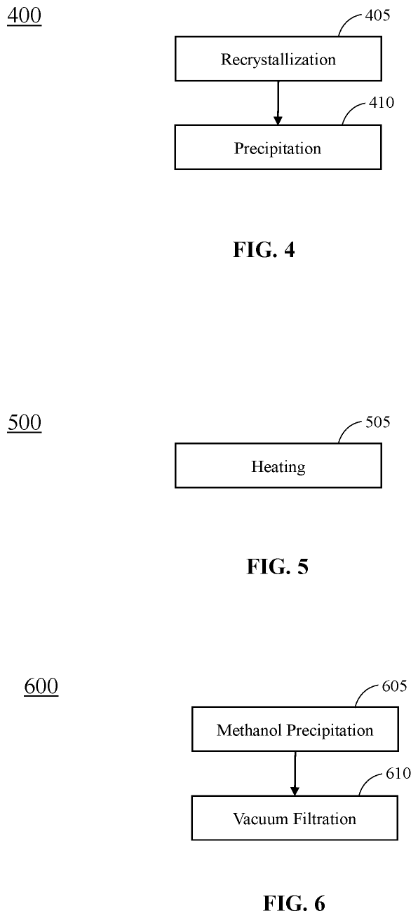

FIG. 4 depicts an illustrative method of producing a recrystallized L-lactide from the L-lactide isomer of method 300, in accordance with present implementations. The method 400 can include recrystallizing the L-lactide isomer via e.g., refrigeration in e.g., ethyl acetate for about e.g., 24 hours at a temperature of about T=3-4° C. (ACT 405). The recrystallized L-lactide can then be precipitated or separated using e.g., vacuum filtration (ACT 410).

FIG. 5 depicts an illustrative method of producing a poly-lactic acid polymer from the recrystallized L-lactide of method 400, in accordance with present implementations. The method 500 can include heating the recrystallized L-lactide of method 400 at e.g., 200° C. for about e.g., 25 minutes under nitrogen. (ACT 505) to produce a poly-lactic acid polymer.

FIG. 6 depicts an illustrative method of producing a purified poly-lactic acid polymer from the poly-lactic acid polymer of method 500, in accordance with present implementations. The method 600 can include precipitating the poly-lactic acid polymer of method 400 in e.g., cold methanol (ACT 605) and filtering the poly-lactic acid polymer (e.g., vacuum filtration) to generate a purified poly-lactic acid polymer. (ACT 610).

FIG. 7 depicts a schematic process for converting post-industrial food waste to biodegradable polymers (i.e., fiber) and textile using the methods disclosed in the present disclosure. The method 700 can include providing a homogenous post-industrial food waste that can be pretreated under acidic or alkali conditions and mechanically processed to improve bioavailability of polysaccharide content. (ACT 705). The method 700 can include combining the pre-treated food waste with an enzyme cocktail to break down polysaccharides for fermentation. (ACT 710). The method 700 can include combining the pre-treated and enzymatically broken-down food waste with microbes in bioreactor for fermentation into bioplastic precursors. (ACT 715). The bioplastic precursors can then undergo heat and pressure treatment in a catalyst-driven polymerization process to generate generic bioplastic. (ACT 720). The generic bioplastic created at the end of ACT 720 is not yet usable in textile manufacturing. The method 700 can include melt-blending four additives with the biopolymer of ACT 720 to optimize 1) thermal resistance, 2) tensile strength and 3) flexibility to par with textile parameters (4) hydrolytic resistance. (ACT 725). The resulting resin (e.g., polymer, plastic alternative, filament, fiber, yarn) is extruded and pelletized. (ACT 725). The method 700 can include dope-dying the yarn (e.g., resin) with appropriate colour and extruding into multifilament fibers using existing polyester machinery. (ACT 730). The method 700 can include weaving, knitting, interlacing, webbing and/or pressing the yarn is into textiles (e.g., fabrics). (ACT 735). Fabrics can either constitute 100% fiber of the biodegradable polymer disclosed herein (e.g, ALT TEX) or can be combined with other fibers, such as cotton to alter fabric properties as needed. (ACT 735).

### Feedstock

In one aspect, the present disclosure provides a method for producing a polymer comprising: providing a feedstock; hydrolyzing the feedstock with an enzyme and fermenting the feedstock with a microbe to produce a mixture; isolating lactic acid from the mixture; converting the lactic acid to poly(lactic) acid; adding, reacting, grafting, and/or cross-linking an additive to the poly(lactic) acid to produce a polymer mixture; forming the polymer with the polymer mixture; and optionally creating a single use plastic alternative, an apparel-grade fabric, fiber, or yarn from the polymer. In some embodiments, the polymer is biodegradable. In some embodiments, the feedstock comprises food waste. In some embodiments, the additive is added to and/or blended with the poly(lactic) acid to produce the polymer mixture. In some embodiments, the additive is reacted with the poly(lactic) acid to produce the polymer mixture. In some embodiments, the additive is grafted to the poly(lactic) acid to produce the polymer mixture. In some embodiments, the additive is crosslinked to the poly(lactic) acid or another polymer to produce the polymer mixture.

In some embodiments, the feedstock used as an input in the method disclosed herein comprises food waste. In any embodiment, the food waste may include carbohydrates including monosaccharides, disaccharides and polysaccharides. Monosaccharides may include dextrose, fructose, galactose, and levulose. Disaccharides may include sucrose, maltose and lactose. Polysaccharides may include starches, dextrins and cellulose. starch-based food waste, fructose-based food waste, cellulose-based food waste, sucrose-based food waste, or combinations of two or more thereof. As used herein, starch-based food waste refers to feedstock in which the majority of polysaccharides present is starch. Non-limiting examples of starch-based food waste include refined grains (e.g. rice, couscous, pastas, noodles, orzo, popcorn, and the like), potatoes (all types, including cull potatoes), corn, peas, squash, breads and bread products, refined cereal, oatmeal, beans, peas, chickpeas, lentils, cornmeal, any type of flour (white, whole what, millet, sorghum, and the like), crackers, chips, baked goods (such as muffins, bagels, and the like), chips (all types), or combinations of two or more thereof. As used herein, fructose-based food waste refers to feedstock in which the majority of saccharides present is fructose. Non-limiting examples of fructose-based food waste include syrups, jam, fruit drinks, fruits (including all pomes, drupes, berries, melons and citrus fruit), root vegetables, or combinations of two or more thereof. As used herein, cellulose-based food waste refers to feedstock in which the majority of polysaccharides present is cellulose. Non-limiting examples of cellulose-based food waste include barley, millet, other grain crop wastes, husks, seed including root vegetables (e.g., yams, beets, parsnips, turnips, rutabagas, carrots, yuca, kohlrabi, onions, garlic, celery root (or celeriac), horseradish, daikon, turmeric, jicama, artichokes, radishes, and ginger, taro, ginseng, and the like), leafy greens (e.g., kale, collard greens, spinach, cabbage, beet greens, watercress, romaine lettuce, arugula, bok choy, sorrel, chard, endive, escarole, microgreens, mustards greens, turnip greens, broccoli, raab, dandelion greens, and the like), seeds including seed husk (e.g., hemp, flax, pumpkin, sunflower, and the like), whole grains (e.g., wheat, barley, brown rice, rye, buckwheat, orzo, spelt, bulgur, millet, durum, quinoa, oats, and the like), beans including bean husk (navy beans, white beans, yellow beans, cranberry, adzuki, french, pinto, mung, black, coffee, and the like), carrots, tomatoes, cucumbers, celery, cereal, split peas, pears, apples, tea leaves, coffee (all parts), or combinations of two or more thereof. In any embodiment, the food waste may include lactose-based food waste.

As used herein, lactose-based food waste refers to feedstock in which the majority of saccharides present is lactose. Non-limiting examples of lactose-based food waste include dairy products (e.g., milk, butter, cheese, yogurt, cottage cheese, sour cream, cream cheese, ice cream, milk powder, and the like). As used herein, sucrose-based food waste refers to feedstock in which the majority of saccharides present is sucrose (i.e., table sugar or cane sugar). Non-limiting examples of sucrose-based food waste include candy, confectionary, molasses, syrups, pastries and baked goods (all types), condiments and sauces, brown sugar, syrups, jam, sugary drinks, or combinations of two or more thereof. In any embodiment, the food waste may include potato waste, coffee waste, apple waste, apple pomace, and/or grain waste. In any embodiment, the food waste may include coffee husk. In any embodiment, the food waste may include potato peel. In any embodiment, the food waste may include coffee husk, apple pomace, and potato peel. In any embodiment, the feedstock may be a non-food feedstock such as paper and/or wood.

As used herein, post industrial food waste is defined as liquid or solid waste generated during any stage of commercial or industrial food or beverage processes (i.e. wasted crops, cull produce, crop byproducts (e.g. begasse), pomace or juice waste, solid food waste after production of food products).

### Hydrolysis

### Enzymes

In some embodiments, the feedstock may be fermented once. In some embodiments, the feedstock may be fermented two or more times. In some embodiments, the feedstock is hydrolyzed with an enzyme. The enzyme may include cellulase, amylase, glucoamylase, beta-glucanase, beta-glucosidase, pectinase, hemicellulase, xylanase, arabanase, pectinase, amyloglucosidase, or combinations of two or more thereof. In any embodiment, the enzyme may include cellulase. In any embodiment, the enzyme may include amylase and glucoamylase.

In some embodiments, the hydrolysis or the fermentation feedstock mixture (for simultaneous saccharification and fermentation) comprises a specified concentration of enzyme. In any embodiment, the mixture may include about 0.1 wt % to about 10 wt % of the enzyme. In any embodiment, the mixture may include about 1 wt % to about 10 wt % of the enzyme. In any embodiment, the mixture may include about 0.5 wt % to about 5 wt % of the enzyme. In any embodiment, the mixture may include about 2 wt % to about 6 wt % of the enzyme. In any embodiment, the mixture may include about 1 wt % to about 3 wt % of the enzyme including about 1.5 wt % to about 2.5 wt %.

In some embodiments, a novel blend of enzymes may be used to hydrolyze the complex polysaccharides. The novel blends of enzymes disclosed herein is catered and customized for each type of food waste input. Alternatively, a novel blend of enzymes disclosed herein may be optimized for various food waste groups/types. The hydrolysis with the novel blend of enzymes may take place a temperature of about 15° C. to about 70° C., optionally at about 50° C.; and pH of about 3 to about 6, optionally at about 5.5. For example, for potato waste hydrolysis, the enzyme blend may include about 1% glucoamylase, about 1% a-amylase, about 0.4% arabanase, about 0.4% cellulase, about 0.4% beta-glucanase, about 0.4% hemicellulase, and about 0.4% xylanase at a temperature of about 50° C. and pH of about 5.5. In some embodiments, the hydrolysis may comprise a specified concentration of enzyme. The hydrolysis or fermentation mixture may be conducted at about 15° C. to about 70° C., optionally at about 50° C.; and pH of about 3 to about 6, optionally at about 5.5.

### Microorganisms

One of the core components of the present disclosure comprises the use of immobilized forms of microbes (e.g., fungal species, NRRL 395 Rhizopus oryzae). This fungal species is specific for L-Lactic acid and produces minimal levels of byproducts. The optimized Rhizopus disclosed herein has been optimized for L-lactic acid yields as high as 135 g/L, whereas the prior art teaches values as high as 100 g/L for semi-continuous or continuous fermentations. The optimized performance is achieved through procedures involving adaptive evolution over 3 years (will continue for longer), genetic modification techniques (i.e. random mutagenesis cycles, shotgun mutagenesis based tools, etc); self-immobilization method/Pellet formation; physical/scaffold immobilization methods (on a scaffold of various shapes and materials); generating more enhanced strain for lactic acid production with optimized conditions. Immobilization is a key factor in increasing yield while the lactic acid yield from the original 40 g/L (not immobilized) value. Whereas the genetic modification and adaptive evolution techniques have helped with yield as well as byproduct reduction (production of bioproducts such as ethanol, fumaric acid, and acetic acid has been minimized). Specifically, the fumaric acid production has been minimized to 0.0 g/L, ethanol to less than 0.6 g/L, and acetic acid to less than 0.01 g/L.

In some embodiments, the feedstock is fermented with a microbe (e.g., a fungus) to produce a mixture comprising lactic acid. In any embodiment, the microbe may include fungi, a bacteria, or a combination thereof. In some embodiments, the microbe may include fungi and/or bacteria. In any embodiment, the microbe may include Aspergillus, Pediococcus, Aerococcus, Carnobacterium, Enterococcus, Tetragenococcus, Vagococcus, Leuconostoc, Oenococcus, Weissella, Streptococcus, Lactococcus, Bacillus, Saccharomyces, Lactobacillus, Rhizopus, or combinations of two more thereof. In any embodiment, the microbe may include Streptococcus bovis, Streptococcus thermophiles, Bacillus coagulans, Saccharomyces cerevisiae, Kluyveromyces lactis, Lactobacillus rhamnosus, Lactobacillus manihotivorans, Lactobacillus plantarum, Lactobacillus paracasei, Lactobacillus delbrueckii subsp. Bulgaricus, Lactococcus lactis, Rhizopus oryzae or combinations of two more thereof. In any embodiment, the microbe may include Bacillus coagulans, Saccharomyces cerevisiae, Streptococcus thermophiles, Lactobacillus rhamnosus, Lactobacillus delbrueckii subsp. Bulgaricus, Lactobacillus plantarum, Rhizopus oryzae, or combinations of two more thereof. In any embodiment, the microbe may include Rhizopus oryzae. In any embodiment, the microbe may include Aspergillus. In some embodiments, the microbe may include one or more Rhizopus oryzae strains and/or subspecies, any species of the Rhizopus genus, or a Rhizopus species selected from Rhizopus arrhizus, delemar, microspores, oligosporus, nodosus, or nigricans.

In any embodiment, the Rhizopus oryzae may include strain NRRL 395[IMI 40564] (e.g, ATCC 9363), ATCC 52311, GY18, NBRC 5384, TISTR 3518, TISTR 3514, TISTR 3523, or combinations of two or more thereof. In any embodiment, the Rhizopus oryzae may include strain NRRL 395. In any embodiment, the Rhizopus oryzae may include strain ATCC 52311. Rhizopus oryzae is a filamentous fungus that belongs to the Zygomycetes family. Rhizopus oryzae is able to produce the sustainable platform chemicals 1-(+)-lactic acid, fumaric acid, and ethanol. During glycolysis, all fermentable carbon sources are metabolized to pyruvate and subsequently distributed over the pathways leading to the formation of these products.

### Immobilization

In some embodiments, the microbe may be mobile. In other embodiments, the microbe may be immobilized (e.g., flocculation, entrapment in a matrix and/or polymer gel, covalently bonded to a carrier, cross-linking of microbial cells, on supports, on scaffolds, biobeads, beads, pellets, carriers, on/within support particles, disc, gel, bed, biopellets). In any embodiment, the immobilization may be via a bead, gel, and/or adsorption on a surface that may comprise a natural carrier such as alginate, carrageenan, guar gum, agar, agarose, chitosan, and/or chitin and/or a synthetic carrier such as acrylamide, polyurethane, polyvinyl, polyvinyl alcohol, polyacrylamide, foam glass, polysulfone matrix, calcium alginate beads, polyisoprene, calcium-alginate-polyvinyl alcohol, pectin, carboxmythyl cellulose crossed linked with ferric/chloride/aluminum chloride, polyvinyl alcohol-sodium alginate-conida, agarose, carboxylmethyl cellulose crossed with metal chlorides or other compounds, phosphorylation beads with other matrix materials, cryogel forms, formaldehyde-urea crosslinked, and/or resin polymers.

In any embodiment, the microbe may be immobilized in/on a bead comprising alginate. In some embodiments, the microbe may be grown or fermented in the presence of a scaffold for cell attachment using materials that include, but are not limited to, activated carbon, wood chips, wooden fibrous networks, wooden sheets, saw dust, bricks, wheat, jute rope, nanosilica, ceramics, cellulose derivatives, cotton cloth/sheet, loofah sponge disks, reticulated foam, coir fibre, loofah sponge, polyurethane, polyurethane foam/bed, polypropylene, polyacrylonitrile, 3D printed bed, washers, honeycomb stainless steel wires, plastic discs, polystyrene mesh/foam beds, polyester-based scaffolds, polyethylene glycol, polyamide, polyethylene, polyvinyl chloride, polystyrene, polychloroprene, cotton, nylon, metal wire or mesh scaffolds (e.g., conformations such as honeycomb, cylindrical, asterisk, hexagonal, octagonal, rod or star formations), or combinations of two or more thereof. In some embodiments, the microbe or cells comprising the microbe may be encapsulated into or mixed with gel to provide beads, droplet, or capsule form. In any embodiment, encapsulation materials may include, but are not limited to, calcium alginate, poly(vinyl alcohol), agar, agarose, polyacrylamide, tetraalkoxysilanes, hydrogels, or combinations of two or more thereof.

The microbe may self-immobilize, flocculate, or form into pellet morphology under certain reactor conditions (e.g., manipulating of reactor pH, agitation speed, aeration rate, baffles, dissolved oxygen, sheer stress, substrate concentration, temperature, carbon:nitrogen ratio in the growth media, and additives including, but not limited to, calcium ions, zinc ions, phosphate, ammonium sulphate, calcium carbonate, ammonium nitrate, chelators, surfactants, polymers, cationic polymers, alginates, biochar, polysaccharides, peptone, corn steep liquor, dried plant matter, starch, powdered food, salts, minerals, clays, bentonite, polygorskite, polyhydroethylmethacrylate, biosurfactant lipids, selenite, nutrients (e.g. KH2PO4, MgSO4, ZnSO4), organic extracts, nanoparticles, microparticles, pectin, metals, ground mycelia mass on agar, yeast forms, and/or soybean peptone.

In any embodiment, the specific pellet-inducing factors may include additives such as polymers, algiantes, starch, bentonite, palygorskite, kaolinite, bisurfactant rhamnolipid, selenite, polyhydroxyethylmethacrylate, maize, pear products, millet, potato, rice polymer, glycerol, salts (KH2PO4, MgSO4, ZnSO4), cationic polymers, polysaccharides, peptone, corn steep liquor, buffered medium, polyoxyethylene sorbitol ester, sorbitols, microparticles (e.g. Al2O3), longan biochar, biocapsule formation medium, ground mycelia mass on agar, yeast extract or yeast forms, etc. In any embodiment, the specific pellet-inducing factors may include conditions such as shear stress, agitation, pH (i.e. low pH), baffles in reactor or flask (decrease size of pellets), etc.

The microbe may be self-immobilized through inoculation in potato dextrose or other broth variations at 5 g/L to 50 g/L CaCO₃, and incubated for 6 hours to 72 hours at 20° C. to 45° C. and 25 rpm-300 rpm. In any embodiment, the microbe may be self-immobilized in pellet formation through inoculation in potato dextrose for 16-24 hours at 30° C. and 200 rpm.

### Genetic Modifications

The microbe disclosed herein may be genetically modified, undergo adaptive evolution, and/or any other modifications to gene and/or protein expression. In any embodiment, the genetic modification, adaptive evolution, or other modification to gene or protein expression may involve methods such as pathway redirection heterologous gene expression, shotgun mutagenesis-based tools, shock-wave mediated transformation, biolistic transformation/biolistics, Agrobacterium-mediated transformation, recombination, viral vectors, selection/selective breeding, chemical mutagenesis (including N-methyl-N′-nitro-N-nitrosoguanidine or diethyl sulfate), induced x-ray mutagenesis, microbial vectors, microprojectile bombardment, electroporation, microinjection, transposons/transposable elements, cloning, homologous recombination, CRISPR, CRISPR/Cas system, transcription activator-like effector nucleases, Cas12a/Cas9-guide RNA system, meganucleases, zinc finder nucleases, transduction, protoplast-mediated transformations, UV mutagenesis, site (PEG)/CaCl2)-mediated protoplast transformation system, site-specific recombination systems (i.e. Cre-loxP), loop-out recombination, replacement-type recombination, protoplastic fusion, agrobacterium-mediated transformation systems (Agrobacterium tumefaciens-mediated transformation (ATMT) system), OE-PCR, entry/gateway cloning, Gibson assembly, BioBricks method, GoldenGate method, SynBac method, Bacteriophage/viral phage genome engineering, modular vector systems, BglBrick method, multiplex automated genome engineering, trackable multiple recombineering, random mutagenesis (i.e. by gamma radiation with 60Co, UV radiation, or low energy ion implantation, etc), double cross-over event for gene knockout, RNA interference for downregulation of gene expression, gene gun.

In any embodiment, the microbe may be genetically modified to produce L-lactic acid. In any embodiment, the microbe may be genetically modified to suppress production of byproducts other than lactic acid. For example, Rhizopus strains that produced 1-(+)-lactic acid contained both lactate dehydrogenase (LDH) (e.g., ldhA and ldhB); while claims that produced fumaric and 1-(+)-malic acid-only contain ldhB. Meussen et al., Appl Microbiol Biotechnol. 94(4): 875-886 (2012).

In any embodiment, the microbe may be genetically modified to produce an increased amount of L-lactic acid compared to the native microbe or enzyme. In any embodiment, the microbe may be genetically modified to withstand varying temperatures, pressure, pH, dissolved oxygen, substrate concentrations, product concentrations, nutrient concentrations, and the like, or combinations of two or more thereof. In any embodiment, the microbe may be genetically modified or stress-adapted to tolerate more acidic pH (e.g., higher lactic acid concentration) to reduce the amount of pH adjusting agent (e.g., alkaline reagents). In any embodiment, the microbe may be genetically modified based on the feedstock to increase and/or optimize lactic acid production (e.g., increase lactic acid concentration and/or increase lactic acid tolerance by at least about 150% including by at least about 200%, at least about 250%, or at least about 300%). Methods for genetically modifying the microbes are disclosed herein.

In any embodiment, the microbe may be subjected to changes in gene/protein expression by adaptive evolution. In any embodiment, the adaptive evolution may include (1) incubating the microbe in a medium comprising one or more sugars (e.g., dextrose) for about 12 hours to about 1 week (including about 24 hours to about 72 hours) to produce an incubated microbe; (2) optionally pretreating the feedstock (e.g., drying, milling, and/or gelatinization); (3) optionally enzymatically hydrolyzing one or more polysaccharides in the feedstock to produce a feedstock hydrolysate; (4) combining the feedstock hydrolysate or the feedstock and one or more growth medium salts (e.g., sulfate, phosphate, hydrogen phosphate, and/or dihydrogen phosphate salts, such as MgSO₄·7H₂O, ZnSO₄·7H₂O, (NH₄)₂SO₄, and/or KH₂PO₄) to produce an adaptation medium; (5) adding the incubated microbe to the adaption medium and incubating for about 12 hours to about 1 week (including about 18 hours to about 72 hours); (6) isolate microbe; and (7) repeat steps 2-6, 3-6, or 4-6 one or more times (e.g., about 1 to about 100 times or more, about 5 to about 50 times, or about 10 to about 40 times) to produce an evolution adapted microbe. In any embodiment, the microbe may include Rhizopus oryzae or a recombinant variant thereof.

### Food Waste Pre-Treatment

In any embodiment, the method may include an acid and/or alkali treatment of the food waste prior to the hydrolyzing and fermenting. Examples of acid treatment include, but are not limited to, dilute acid treatment such as dilute sulphuric acid treatment. Examples of alkali treatment include, but are not limited to, alkali treatment such as KOH, NaOH, and/or Ca(OH)₂ treatment such as 2.5-50 g/L. As used here, acidic treatment refers a pH at or below 6 (more preferably 5) and alkali treatment refers to a pH at or above 8 (more preferably 9).

In any embodiment, the method may include pre-treating the feedstock by steam explosion or by adding a solvent at a temperature above about 60° C. In any embodiment, the solvent may include water having a pH of about 6.5 to about 7.5 including about 6.8 to about 7.2 or about 6.9 to about 7.1. In any embodiment, the solvent may include water having a pH of about 3.5 to about 7.5 including about 5.5 to about 7.2 or about 6.9 to about 7.1. In any embodiment, the temperature may be about 70° C. to about 110° C. In any embodiment, the temperature may be about 70° C. to about 120° C. In any embodiment, the pre-treating may occur for at least about 20 minutes. In any embodiment, the pre-treating may occur for about 30 minutes to about 90 minutes. In any embodiment, the pre-treating may occur for about 30 minutes to about 300 minutes.

In any embodiment, the microbe may be added to a composition comprising the feedstock and the enzyme. Alternatively, the enzyme may be added to a composition comprising the feedstock and the microbe. Alternatively, the feedstock may be added to a composition comprising the enzyme and the microbe. The hydrolysis and fermentation can occur in same vessel or different vessels. In some embodiments, the hydrolyzing may occur prior to the fermenting. In other embodiments, the hydrolyzing and fermenting may occur simultaneously.

In any embodiment, the hydrolyzing and/or fermenting may occur via a one-pot reaction (i.e., no work-up or purification after hydrolyzing and before fermenting or no work-up or purification after hydrolyzing and before fermentation). In any embodiment, the hydrolyzing and/or fermenting may be conducted by a batch, fed-batch, repeated-batch, semi-continuous, or continuous process. In any embodiment, the hydrolyzing and/or fermenting may be conducted by fed-batch approach. In that embodiments, the hydrolyzing and/or fermenting may be conducted over 65 hours and/or 4 additions of glucose salt media comprising varying concentrations (e.g., about 10 g/L to about 300 g/L, or about 10 g/L to about 1000 g/L). Alternatively, the hydrolyzing and/or fermenting may be conducted over 16 to 96 hours (or semi-continuous/continuous) and/or 1-104 additions of glucose salt media comprising varying concentrations (e.g., about 10 g/L to about 1000 g/L). In that embodiments, the hydrolyzing and/or fermenting may be conducted over 65 hours and/or 4 additions of glucose salt media comprising varying concentrations (e.g., about 300 g/L to 600 g/L). In any embodiment, the microbe (mobile or immobile) may be static, rotating, free-floating, or a combination of two or more thereof in the reactor.

In any embodiment, the microbe may be in a reactor with a fluidized bed, static bed, rotating biological contactor, rotary biofilm contactor, rotating fibrous bed bioreactor, air-lift, fixed, bed, static bed, column, packed column, packed-bed/packed bed column, stirred tank reactor, hybrid designs, membrane bioreactor, aerated bioreactor, air lift bioreactor, airlift reactor with net draft tube, biosorption column, bubble column bioreactor, chemostat, column reactor, flasks, shake flasks, screw cap bottle shaker, solid-phase extraction, stirred-tank reactor, and three-phase fluidized bed reactor, extractive fermentation bioreactors, and/or any combination of the above.

In any embodiment, the hydrolyzing the feedstock with an enzyme and/or the fermenting the feedstock with a microbe to produce a mixture may be conducted at a temperature between about 10° C. and about 75° C. including a temperature between about 40° C. and about 60° C. or between about 15° C. and about 35° C. In any embodiment, the hydrolyzing the feedstock with an enzyme and/or the fermenting the feedstock with a microbe to produce a mixture may be conducted at about 30° C. to about 35° C. In a preferred embodiments, the hydrolyzing the feedstock with an enzyme and/or the fermenting the feedstock with a microbe to produce a mixture may be conducted at about 30° C. In any embodiment, the hydrolyzing and/or fermenting may be conducted at a pH between about 4 to about 8 including about 5.5 to about 7.5, or about 6 to about 7. In any embodiment, the hydrolyzing and/or fermenting may be conducted at a pH between about 3 to about 8 including about 5.5 and about 7.5 or about 6 to about 7.

The enzyme to microbe ratio may impact the yield of lactic acid generated using the method disclosed herein. In any embodiment, the weight ratio of the enzyme to the microbe may be about 10:1 to about 1:10. In any embodiment, the weight ratio of the enzyme to the microbe may be about 5:1 to about 1:5. In any embodiment, the weight ratio of the enzyme to the microbe may be about 3:1 to about 1:3. In any embodiment, the weight ratio of the enzyme to the microbe may be about 2:1 to about 1:2. In any embodiment, the weight ratio of the enzyme to the microbe may be about 1:1 to about 1:1.

### pH Adjustment

In some cases, the alkaline source used to adjust the pH of the fermented feedstock mixture enhances the yield of the lactic acid generated using the methods disclosed herein. The industry alkaline source standard is CaCO₃. However, addition of CaCO₃ to a feedstock fermentation mixture generates significant amount of toxic byproducts, such as for example calcium sulphate (also known as gypsum). In some embodiments, for eco-friendly procedure and non-toxic byproducts, MgO and Mg(OH)₂ based pH control, which are the least common alkaline sources, are utilized in the method disclosed herein.

In any embodiment, the method may further include adding a pH adjusting agent following the hydrolyzing and fermenting. In that embodiment, the fermentation mixture may be agitated at 200 rpm and/or 2 vvm max under aeration. In any embodiment, the fermentation mixture may be agitated at 200 rpm and/or aerated with conditions of 2 vvm. In any embodiment, the fermentation mixture may be agitated at 50-350 rpm and/or aerated with conditions of 0.5-10 vvm. In any embodiment, the fermentation mixture may be agitated at 200 rpm and/or aerated with conditions of 2 vvm. The pH adjusting agent may be any known basic pH adjusting agent such as a carbonate (e.g., CaCO₃), bicarbonate (e.g., NaHCO₃), hydroxide (e.g., KOH, NaOH, Al(OH)₃, Ca(OH)₂, Mg(OH)₂, and/or NH₄OH), amines (e.g., NH₃), oxides (e.g., MgO), non-nucleophilic bases or combinations of two or more thereof. In any embodiment, the pH adjusting agent may include calcium carbonate. In any embodiment, the pH adjusting agent may include NaOH, Mg(OH)₂, MgO, and/or NH₄OH. In any embodiment, the pH adjusting agent may include NaOH. In any embodiment, the pH adjusting agent may include MgO, optionally 1.5M or any concentration that will dissolve in the mixture. In any embodiment, the pH adjusting agent may include Mg(OH)₂. In any embodiment, the pH adjusting agent may include MgO and Mg(OH)₂. In that embodiment, MgO and/or Mg(OH)₂ may enhance or increase the yield of lactic acid isolated from the fermentation mixture when compared to the yield generated with, for example, CaCO₃.

### Lactic Acid Isolation and Conversion

The L-lactic contained in the fermentation mixture may be purified using purification methods known to a person of skill in the art. For example, the L-lactic may be purified via centrifugation, activated carbon use, filtration, rotary evaporation, and ionic exchange chromatography, or liquid-liquid extraction (simple or reactive extractions). Ionic exchange chromatography has not been used at scale in the industry, as liquid extraction mechanisms are industry standards. Accordingly, in some embodiments, the isolating the lactic acid may include ionic exchange chromatography with acid or base elutions, and cation removal resins. In any embodiment, the lactic acid may be extracted with an organic solvent comprising ethyl acetate. In any embodiment, the lactic acid may be extracted with an organic solvent comprising ethyl acetate and ionic exchange chromatography with acid or base elutions, and cation removal resins.

In any embodiment, the isolating the lactic acid may include clarifying (e.g., centrifuge. decanter, disk-stack bowls), flocculation (e.g. with polymeric resins, chitosan, polyacrylamide, other charged particles), filtering (e.g. depth, microfiltration, vacuum, gravity, pressure, cross-filtration, tangential flow filtration, rotary drum filtration, nanofiltration, ultrafiltration, reverse-osmosis, hollow-fibre, and/or membrane filtration), concentrating (e.g., distillation and/or rotary evaporation), extracting (e.g., organic solvents such as alcohols (e.g. methanol, ethanol, n-propanol, isopropanol, n-butanol, 2-ethylhexanol, n-octanol, butan-2-ol, n-decanol), ethyl methyl ketone, dimethylformamide, diisobutylketone, Diethylsabecate, dichloromethane, 1,4-dioxane, hexane, n-decane, n-octane, n-undecane, isobutyl methyl ketone, ketones, methyl-tertiary-butylether (MIBK, methyl-isobutyl-ketone), methyl-tertiary-butylether, chloroform, kerosene, toluene, methylene chloride, diethyl ether, tetrahydrofuran, ethyl acetate, 2-methylTHF, tributylphosphate, 1,8-cineol, CPME (cyclopentyl methyl ether), trialkyl phosphine oxide (with or without sulfonated kerosene), isomyl alcohol, acetone, amines (e.g. triethylamine, trioctylamine, tributylamine), trioctylmethylammonium chloride (octane at various concentrations, and varying polysorbate-80), oils (e.g. sunflower oil, cotton seed oil), ionic liquids, supercritical fluids, or combinations of two or more thereof).

In any embodiment, the isolating the lactic acid may include distilling, adding activated carbon and filtering, sonication, cross-filtration, chromatography (Preparative HPLC, column chromatography, thin layer chromatography, Silica Gel Column Chromatography, Flash Chromatography, Supercritical Fluid Chromatography, Reverse-Phase HPLC, Size exclusion chromatography, Simulated Moving Bed (SMB) chromatography), precipitation (via addition of reagents including/but not limited to acids for acidification), salting-out extraction, emulsion liquid membrane extraction, molecular distillation, pervaporation esterification, esterification, reactive distillation, extraction using extractants (e.g., trioctylamine, tri-n-decylamine, tri-octyl/decyl amine in methyl isobutyl ketone and chloroform, diisopropyether, ethyl acetate, diethyl ether, chloroform, pentane, hexane, toluene, amines (e.g. trioctylamine, tributylamine, tri-n-octylamine, dioctylamine), tributyl phosphate, tri-n-octylphosphine oxide, isopentyl alcohol), electrodialysis (e.g., watersplitting electrodialysis), adsorption, membrane or electromembrane separation (e.g., ion exchange membranes, bipolar membranes, electrometathesis, electrodeionization, electro-ion substitution, electrodeionization, electrodialysis, watersplitting electrodialysis, bipolar electrodialysis, salt-splitting electrodialysis, etc), liquid-liquid extraction, solid-liquid extraction, decolorization, or combinations of two or more thereof. In any embodiment, the lactic acid may be extracted with an organic solvent comprising trialkyl phosphine oxide. In any embodiment, the lactic acid may be extracted with an organic solvent comprising isoamyl alcohol. In any embodiment, the lactic acid may be extracted with an organic solvent comprising ethyl acetate.

In any embodiment, the lactic acid may be separated/purified from broth via ionic exchange chromatography (i.e. strong or weak anionic and/or cationic resins) at various pH, substrate concentrations, temperatures, resin materials, flow rates (bed volumes per hour), solvent concentrations, and lactic acid (LA):resin ratios. In any embodiment, the other byproducts or impurities may be removed via ionic exchange chromatography (i.e. strong or weak anionic and/or cationic resins). In any embodiment, the ionic exchange chromatography may involve weak and/or strong anion and cation exchange resins made of materials such as acrylic, polymer, styrene-divinylbenzene (gel), styrene-divinylbenzene (macroreticular), hyper-crosslinked polystyrene divinylbenzene, polystyrene, gel, crosslinked acrylic acid copolymer, etc. It can involve the use of solvents (or various concentrations) such as HCl, NaOH, methanol for elution and/or resin regeneration. In any embodiment, the ionic exchange chromatography may involve flow rates of 0.25-25 Bed volumes/hour with L-LA: resin ratio of 10:1 to 1:10. For example, the present invention may conduct gel-based strong acid cation exchange resin to convert dissociated lactic acid in fermentation broth into undissociated form, followed by acrylic-gel weak base anion exchange resin chromatography to separate lactic acid from the fermentation impurities at flow rates of 12 Bed volumes/hour with L-LA:resin ratio of 1:2 with use of HCl or NaOH for elution.

In any embodiment, the isolating the lactic acid may occur during or after the hydrolyzing and/or fermenting. In any embodiment, the isolating the lactic acid may occur during or after the fermenting. In any embodiment, the isolating the lactic acid may occur during the fermenting using a 1, 2, or 3-phase technique.

In any embodiment, the lactic acid includes L-lactic acid. In any embodiment, the lactic acid includes at least about 50 wt % of the L-lactic acid. In any embodiment, the lactic acid includes about 75 wt % to about 100 wt % of the L-lactic acid. In any embodiment, the lactic acid is substantially free of D-lactic acid.

In another embodiment, the lactic acid includes D-lactic acid. The lactic acid may include at least about 50 wt % of the D-lactic acid such as about 75 wt % to about 100 wt % of the D-lactic acid. The lactic acid may be substantially free of L-lactic acid.

In some embodiments, the purified lactic acid (e.g., L-lactic acid) is converted to Lactide via dehydration, oligomerization, and depolymerization as known to a person of skill in the art. In some embodiments, the reaction conditions, such as temperature, pressure, atmospheric conditions are customized and/or optimized for foodwaste derived lactic acid as disclosed herein.

In any embodiment, the converting the lactic acid to the poly(lactic) acid may include an azeotropic dehydrative condensation process, a direct polycondensation process, depolymerization process, and/or a or a lactide synthesis followed by ring opening polymerization process.

In any embodiment, the converting the lactic acid to the poly(lactic) acid may include dehydration of the lactic acid. In any embodiment, the converting the lactic acid to the poly(lactic) acid may include oligomerization of dehydrated lactic acid. In any embodiment, the converting the lactic acid to the poly(lactic) acid may include a depolymerization of the oligo(lactic acid)/oligomerized lactic acid/OLA into cyclic lactide (e.g., via formation of lactide stereoisomers such as meso-lactide, D-lactide and/or L-lactide). In any embodiment, the converting the lactic acid to the poly(lactic) acid may include ring opening polymerization process to convert lactide into PLA (i.e. polymerizing the cyclic lactide to produce the poly(lactic) acid.

In any embodiment, the converting the lactic acid to the poly(lactic) acid may include a ring opening polymerization process. In any embodiment, the ring opening polymerization process may include converting the lactic acid to oligo(lactic acid), depolymerizing the oligo(lactic acid) to produce cyclic lactide (e.g., formation of lactide stereoisomers such as meso-lactide, D-lactide and/or L-lactide), and polymerizing the cyclic lactide to produce the poly(lactic) acid via ring-opening polymerization mechanisms.

In any embodiment, lactic acid may be converted to dehydrated lactic acid. In any embodiment, the conversion of lactic acid to dehydrated lactic acid may include heating and/or dehydrating (i.e., removing water). In any embodiment, the converting the lactic acid to the oligo(lactic acid) may include heating and/or dehydrating (i.e., removing water). In any embodiment, the heating may be at or above about 100° C. In any embodiment, the heating may be between about 100° C. and about 200° C., about 120° C. and about 180° C., about 140° C. and about 160° C., or about 145° C. and about 155° C. In any embodiment, the dehydrating may be at or above about 50° C. In any embodiment, the dehydrating may be between about 50° C. and about 100° C., about 55° C. and about 90° C., about 60° C. and about 80° C., or about 70° C. and about 85° C.

In any embodiment, the heating and/or dehydrating may be at a pressure less than standard atmosphere (i.e., less than 101,325 Pa). In any embodiment, the pressure may be between about 100 Pa to about 50,000 Pa, about 1000 Pa to about 20,000 Pa, about 2000 Pa to about 10,000 Pa, about 3000 Pa to about 5000 Pa, about 5000 Pa to about 7500 Pa, or about 3000 Pa to about 7500 Pa. In some embodiments, the pressure is reduced from 7500 Pa to 4500 Pa during dehydration. In any embodiment, the heating and/or dehydrating may be conducted under an inert gas (e.g., N₂ or argon) or under atmospheric conditions (e.g., air). In any embodiment, the heating and/or dehydrating may occur for about 1 hour to about 10 hours, about 2 hours to about 8 hours, or about 4 hours to about 6 hours. In any embodiment, the heating and/or dehydrating may occur for about 1 hour, about 1.5 hours, about 2 hours, about 3 hours, about 4 hours, about 5 hours, about 6 hours, about 7 hours, about 8 hours, about 9 hours, or about 10 hours.

In some embodiments, the dehydrated lactic acid is oligomerized to form oligo(lactic acid) or oligomers of lactic acid. In any embodiment, the oligomerization of the dehydrated lactic acid to form oligo(lactic acid) or oligomers of lactic acid may include heating, dehydrating and/or condensation reactions (i.e., removing water). In any embodiment, the heating may be at or above about 100° C. In any embodiment, the heating may be between about 100° C. to about 210, about 110° C. to about 200° C., about 120° C. to about 190° C., or about 140° C. to about 185° C. In any embodiment, the temperature is about 100° C., about 110° C., about 120° C., about 130° C., about 150° C., about 160° C., about 170° C., about 180° C., about 190° C., about 200° C., about 210° C., about 220° C., about 230° C., about 240° C., about 250° C., about 260° C., about 270° C., about 275° C., about 280° C., about 290° C., or about 300° C.

In any embodiment, the pressure may be between about complete vacuum (0 Pa) to about 50,000 Pa, about 100 Pa to about 50,000 Pa, about 500 Pa to about 20,000 Pa, about 1000 Pa to about 15,000 Pa, or about 1500 Pa to about 9000 Pa. In any embodiment, the heating and/or dehydrating may be conducted under an inert gas (e.g., N₂ or argon) or atmospheric conditions (e.g., air). In any embodiment, the oligomerization may occur for about 1 hour to about 10 hours, about 2 hours to about 8 hours, or about 2 hours to about 7 hours. In any embodiment, the heating and/or dehydrating may occur for about 1 hour, about 1.5 hours, about 2 hours, about 3 hours, about 4 hours, about 5 hours, about 6 hours, about 7 hours, about 8 hours, about 9 hours, or about 10 hours.

In any embodiment, the converting the lactic acid to the poly(lactic) acid may include a depolymerization process. In any embodiment, the depolymerizing the oligo(lactic acid) to produce cyclic lactide may include distilling. In any embodiment, the distilling may be at a temperature above about 100° C. In any embodiment, the temperature is between about 120° C. and about 275° C., about 150° C. and about 220° C., about 170° C. and about 220° C., or about 190° C. and about 200° C. In any embodiment, the temperature is about 100° C., about 110° C., about 120° C., about 130° C., about 150° C., about 160° C., about 170° C., about 180° C., about 190° C., about 200° C., about 210° C., about 220° C., about 230° C., about 240° C., about 250° C., about 260° C., about 270° C., about 275° C., about 280° C., about 290° C., or about 300° C.

In any embodiment, the depolymerization reaction may be at a pressure less than standard atmosphere (i.e., less than 101,325 Pa). In any embodiment, the pressure may be between about 50 Pa to about 35,000 Pa, about 100 Pa to about 1000 Pa, about 200 Pa to about 800 Pa, about 100 Pa to about 15,000 Pa, about 200 Pa and about 10,000 Pa, about 500 Pa and about 7500 Pa, or about 300 Pa to about 500 Pa. In any embodiment, the pressure may be between about complete vacuum (0 Pa) to about 35,000 Pa, about 100 Pa to about 10,000 Pa, about 200 Pa to about 8000 Pa, about 300 Pa to about 5,000 Pa, about 500 Pa and about 3000 Pa, about 750 Pa and about 1500 Pa, or about 800 Pa to about 1000 Pa. In any embodiment, the distilling may occur for about 1 hour to about 10 hours, about 1 hour to about 12 hours, about 1.5 hours to about 8 hours, about 2.5 hours to about 6 hours, or about 2.5 hours to about 4 hours. In any embodiment, the distilling may occur for about 1 hour, about 1.5 hours, about 2 hours, about 3 hours, about 4 hours, about 5 hours, about 6 hours, about 7 hours, about 8 hours, about 9 hours, about 10 hours, about 11 hours, or about 12 hours.

### Catalysts

The catalyst type used in the depolymerization and/or the Ring-opening polymerization step of the method disclosed herein is an important part of the technology disclosed herein. In any embodiment, the distilling may include a rectification column. In any embodiment, the depolymerization or ring-opening polymerization may include a reactor (glass, steel etc). In any embodiment, the depolymerizing the oligo(lactic acid) to produce cyclic lactide may include adding a catalyst (e.g., Zn(La)₂, creatinine, sodium bicarbonate, Sn(OEt)₂, ZnO (or various particle sizes, TnO, Zn acetate, SnO, Al2SO3, SiO, SnCl2, Sb2O3, SnHPO3, Tin powder, tin alkoxides, tin halides, SnO2, Pb oxide, beta zeolites, sulfuric acid, SnHPO3, CsCO3, ZIF, or combinations of two or more thereof). In any embodiment, the catalyst may include Zn(La)₂ and sodium bicarbonate. In any embodiment, the catalyst may include zinc oxide, such as a zinc oxide powder (or various particle sizes) In any embodiment, the monomer:catalyst ratio may be between 0.0001% to 10%. For example, the catalyst blend can include 0.5% Zn(La)₂ and 0.2% sodium bicarbonate. Another example, the catalyst blend includes 0.6% ZnO and 0.4% sodium bicarbonate.

In any embodiment, the reaction(s) (i.e. dehydration, oligomerization, depolymerization) may be conducted in a reactor as a separate steps, as one-pot reaction, multiple reactors, rotary evaporator, etc. In any embodiment, the cyclic lactide may be crystallized one or more times prior to the polymerizing. In any embodiment, the cyclic lactide may be crystallized one or more times using ethyl acetate, toluene, or another suitable solvent/combination thereof. In any embodiment, the crystallizing may include temperatures below room temperature (e.g., −15 C to about 15° C., 0 to about 15° C. or about 2° C. to about 5° C.) and/or a time of about 8 hours to about 3 days (e.g., about 12 hours to about 48 hours or about 20 hours to about 30 hours).

In any embodiment, the cyclic lactide may be crystallized one or more times prior to the polymerizing. In any embodiment, the cyclic lactide may be crystallized one or more times using ethyl acetate, toluene, or another suitable solvent. In any embodiment, the crystallizing may include temperatures below room temperature (e.g., 0 to about 15° C. or about 2° C. to about 5° C.) and/or a time of about 8 hours to about 3 days (e.g., about 12 hours to about 48 hours or about 20 hours to about 30 hours).

### Poly-Lactic Acid Polymer

In some embodiments of the method disclosed herein, the poly(lactic) acid is produced by polymerizing the cyclic lactide. In any embodiment, the polymerizing the cyclic lactide to produce the poly(lactic) acid may include heating. In any embodiment, the catalyst is added directly, or dissolved in solvent, or supported on a polymer matrix. In any embodiment, the heating may be at a temperature above about 120° C. In any embodiment, the temperature is between about 120° C. and about 300° C., about 130° C. and about 250° C., about 150° C. and about 220° C., about 180° C. and about 220° C., or about 190° C. and about 210° C. In any embodiment, the heating may be at a temperature above about 100° C. In any embodiment, the temperature is between about 100° C. to about 300° C., about 110° C. to about 250° C., about 120° C. to about 220° C., about 140° C. to about 220° C., or about 160° C. to about 210° C. In any embodiment, the temperature is about 125° C., about 130° C., about 150° C., about 160° C., about 170° C., about 180° C., about 190° C., about 200° C., about 210° C., about 220° C., about 230° C., about 240° C., about 250° C., about 260° C., about 270° C., about 275° C., about 280° C., about 290° C., or about 300° C.

In any embodiment, the heating may be conducted under an inert gas (e.g., N₂ or argon). In any embodiment, the heating may occur for about 5 minutes to about 5 hours, about 10 minutes to about 2 hours, or 20 minutes to about 1 hour. In any embodiment, the heating may occur for about 5 minutes to about 10 hours, for about 5 minutes to about 12 hours, about 10 minutes to about 5 hours, or 20 minutes to about 4 hour. In any embodiment, the heating may occur for about 5 minutes, about 10 minutes, about 15 minutes, about 20 minutes, about 30 minutes, about 40 minutes, about 60 minutes, about 60 minutes, about 90 minutes, about 100 minutes, about 120 minutes, about 150 minutes, about 180 minutes, about 210 minutes, about 240 minutes, about 270 minutes, or about 300 minutes. In any embodiment, the polymerizing may occur with or without solvents, such as toluene, which can be replenished throughout the reactions.

In any embodiment, the polymerizing may further include adding a catalyst. The catalyst may include Ca(OMe)₂, (C₉H₂₁O₃)Y, Al(OiPr)₃, Sn(Oct)₂, ZnO, Zn acetate, Zn(lactate), aluminum isopropoxide, lanthanum isopropoxide, tin isopropoxide, Guanidinate Zn(II) complex, Zinc complexes with any R groups, Calcium complexes with any R group ligands. Barium complexes with any R group ligands, strontium complexes with any R groups, tin complexes with any R group ligands, germanium complexes with any R group ligands, copper complexes with any R group ligands, Zirconium complexes with any R group ligands, titanium complexes with any R group ligands, aluminum triisopropoxide, salen metal alkoxides, DBU, bismuth subsalicylate, bismuth (III) acetate, bismuth methylene, Bismuth lactate, bismuth complexes (with any R groups), Iron subsalicylate, Aluminum subsalicylate, Iron octoate, copper octoate, zinc octoate, zinc lactate, iron chlorides, creatinine glycolate/acetate, thiozole carbenes, imidozolium carbenes, imidazolium carbenes, pyridine-derived bases (e.g. DMAP (4-(dimethylamino)-pyridine), DMAP-DCC, DMAP-MSA, PPY (4-(dimethylamino)-pyridine)), guanidine and amidine derivatives (e.g. 1,5,7-tri-azabicyclododecene (TBD), N-methyl-1,5,7-triazabicyclododecene (MTBD), di-azabicycloundecene (DBU), N-heterocyclic carbenes, phosphazenes, B-ketoiminate, Zn phenoxide, Fe(III) alkoxide, Di-zinc complex, or combinations of two or more thereof. In any embodiment, the polymerizing may further include addition of initiator if necessary, such asI-dodecanol thiol urea, pentadiol, triethylene glycol. EG, PEG variaties, PGA, pyrenebutanol, amines, alcohols of varying lengths and functional groups (diols. triols, tetra-ols)), TiO2, or combinations of two or more thereof.

In any embodiment, the catalyst may include organic catalysts or metal-based catalyst (e.g., metal containing catalyst). In any embodiment, the organic catalyst may be selected from creatinine, creatinine alcohol, creatinine alkyl adducts, glycolate, acetate, thiozole carbenes, imidozolium carbenes, imidazolium carbenes, pyridine-derived bases (e.g. DMAP (4-(dimethylamino)-pyridine), DMAP-DCC, DMAP-MSA, PPY (4-(dimethylamino)-pyridine)), guanidine and amidine derivatives (e.g. 1,5,7-tri-azabicyclododecene (TBD), N-methyl-1,5,7-triazabicyclododecene (MTBD), di-azabicycloundecene (DBU), any N-heterocyclic carbenes, phosphazenes, trifluoromethylsulfonic acid, thioureas, 1,1,2,2,3,3-hexa-alkylguanidinium acetate (HAG ? OAc), creatinine acetate, triazol-5-ylidenes and their alcohol and alkyl adducts, 1,3-dimesitylimidazolin-2-ylidene and its alcohol adducts, or 1,5,7-triazabicyclo[4.4.0]dec-5-ene (TBD) and its alcohol adducts.

The metal-based catalyst may be selected from a didentate, tridentate, or tetradentate catalyst. The metal-based catalyst can be mononuclear, dinuclear, trinuclear, or tetranuclear. The catalyst can be a metal core coordinated with ligands. The metal can be selected from the group consisting of calcium, magnesium, aluminum, tin, zinc, lanthanum, barium, copper, germanium, strontium, bismuth, zirconium, titanium, iron, gallium, cobalt, chromium, indium, hafnium, sodium, potassium, yttrium, cesium, and other metals. The ligands can be include any β-diketiminate ligands, any salen ligand, any tert-butyl hydroxytoluene (BHT) ligands, any pyrazolonate ligands, any amino phenolate ligands, borate ligands such as tris(pyrazolyl)borates and tris(indazolyl)borate, any N-heterocyclic carbene ligands, any phenoxy ligands, silane ligands such as trimethylsilane, tetrahydrofuran ligands, furan ligands, porphyrin ligands with subsituents, any pyrrolidine ligands and pyrrolidine ligands with substituents, pyridine ligands and pyridine ligands with substituents, pyrrole ligands and pyrrole ligands with substituents, imidozolidine ligands, imidazolidine lignads, imidazole ligands, phosphoimine ligands, pyrazolidine ligands and those with substituents, pyrazole ligands and those with substituents, oxazole and oxazolidine ligands and those with substituents, isoxazolidine and isoxazole ligands and those with substituents, thioazole ligands and those with substituents, thiazolidine ligands and those with substituents, triazole ligands, any oxadiazole ligands and those with substituents, furazan ligands and those with substituents, piperidine ligands and those with substituents, any oxane ligands with substituents, and pyran ligands and those with substituents, any diazinane ligands and those with substituents, any diazine lignads and those with substituents, any morpholine ligands and those with substituents, any oxazine ligands and those with substituents, any thiazine ligands and those with substituents, any thiomorphorline ligands and those with substituents, any dioxane ligands and those with substituents, any dioxine ligands and those with substituents, any trioxane ligands and those with substituents, any triazinane ligands and those with substituents, any crown ether ligands, any amine ligands, alkoxy ligand, alkyl ligand, phenol ligands, amidinate ligands, aldiminate ligands, guandinate ligands, Schiff base or imine ligands, iminopyridine ligands, scorpionate ligands. The ligands, and/or the metal-ligand complex might be chiral or achiral.

In any embodiment, the catalyst may be a metal salt. The salt may contain a cation and/or an anion. The cation may be calcium, magnesium, aluminum, tin, zinc, lanthanum, barium, copper, germanium, strontium, bismuth, zirconium, titanium, iron, sodium, potassium, yttrium, and other metals. The anion may be any halides, alkoxides, phenoxides, carbonate, carboxylates (octanoates, acetates, subsalicylates, trifluoroacetates, isobutyrates, etc.).

In any embodiment, the catalyst may be a bifunctional catalyst system using a hydrogen bond donor and a hydrogen bond acceptor. In any embodiment, the hydrogen bond donor might be phenols, bis-sulfonamides, fluorinated diols, amido-indole, thiol urea, squaramides, cationic amines and cationic guanadines, solvent-separated ion pairs facilitated by crown ethers, α-halogenoacetanilides, azophosphatrane, and phosphoric acids. In any embodiment, the hydrogen bond acceptor might include sparteine or dimethylaminopyridine (DMAP). In any embodiment, the catalyst system may involve a combination of two or more of the catalysts disclosed herein.

In any embodiment, the polymerization may involve the use of an initiator. The initiator may include alcohols of varying lengths and functional groups (diols. triols, tetra-ols) such as dodecanol, pentadiol, triethylene glycol. EG, PEG with varying molecular weights, PEG copolymers or any PEG derivatives, PGA, pyrene butanol, benzyl alcohol. The initiator may include any amines, thiourea. In any embodiment, the initiation system may involve a combination of two or more of the initiators above. In any embodiment, the post-polymerization crude or purified polymer may undergo further reaction such as chain-end protection (e.g. using acetic anhydride, acyl chloride) to protect alcohol groups, acid treatment (e.g. HCl, H2SO4, acidic methanol) at varying concentration to remove residual catalyst, alcohol group protection (e.g. treatment with any silanes, silyl ethers such trimethyl silyl chloride), crosslinking (e.g. 1,2,3,4-butanetetracarboxylic, 1,4-butanedicarboxylic acid, 1,3-propanetricarboxylic acid, carbodiimides, DCC (dicyclohexylcarbodiimide), EDC (1-ethyl-3-(3-dimethyaminopropylcarbodiimide)), ZIKA (bis(2,6-diisopropylphenyl)carbodiimide)), and/or antihydrolysis agent reactions (e.g. bis(2,6-diisopropylphenyl)carbodiimide (BDICDI)).

In any embodiment, the method may further include purifying the poly(lactic) acid. In any embodiment, the purifying may include dissolving and precipitating the poly(lactic) acid followed by filtering. In any embodiment, the dissolving and precipitating may be conducted with an organic solvent (e.g., chloroform, dichloromethane, tetrahydrofuran, hexofluoroisopropanol. C1-C5 alcohol such asmethanol, methanol (+other alcohols), diethyl ether, water, hexane (+other alkanes), petroleum ether). In any embodiment, the purification process may be involve dissolving polymer in chloroform completely at varying temperatures and precipitating using methanol. In any embodiment, the purification may involve rapid purification cycles involving submerging, partially dissolving, and stirring the crude polymers in combined mixtures of chloroform:methanol at ratios of 5:1 to 1:5. In any embodiment, the purification may involve rapid purification cycles involving submerging, partially dissolving, and/or stirring the crude polymers in combined mixtures of chloroform:methanol at ratios of 6:1 to 1:6. In any embodiment, the purification protocol may involve submerging or partially dissolving the crude polymers in chloroform:methanol mixture ratio of 1:4 and heating at about 50° C. to about 70° C. for about 15 mins to about 180 mins. In any embodiment, the purification processes can occur at temperatures from about 0° C. to about 100° C. for about 5 mins to about 6 hours with or without stirring/agitation. In any embodiment, the purification protocol may involve submerging or partially dissolving the crude polymers in chloroform:methanol mixture ratio of 1:4 and heating at about 60° C. for about 30 mins with stirring.

In any embodiment, the poly(lactic) acid may have a molecular weight (MN) of about 20,000 to about 500,000, about 30,000 to about 300,000, about 50,000 to about 250,000, or about 60,000 to about 200,000. In any embodiment, the poly(lactic) acid may have a molecular weight (MN) of about 90,000 to about 160,000. In any embodiment, the poly(lactic) acid may have a molecular weight (MN) of about 90,000, about 100,000, about 110,000, about 120,000, about 130,000, about 140,000, about 150,000, about 160,000, about 170,000, about 180,000, about 190,000, about 200,000, about 210,000, about 220,000, about 230,000, about 240,000, about 250,000, about 260,000, about 270,000, about 280,000, about 290,000, or about 300,000.

In any embodiment, the polymer may exhibit a tensile strength of about 30 MPa to about 90 MPa. In any embodiment, the polymer may exhibit a tensile strength of about 50 MPa to about 75 MPa. In any embodiment, the polymer may exhibit a tensile strength of about 30, about 40, about 50, about 60, about 70, about 80, about 90, about 100, about 125, about 150, about 175, about 200 MPa. In any embodiment, the polymer may exhibit a Young modulus of about 500 MPa to about 2700 MPa. In any embodiment, the polymer may exhibit a Young modulus of about 500 MPa, about 600 MPa, about 700 MPa, about 800 MPa, about 900 MPa, about 1000 MPa, about 1100 MPa, about 1200 MPa, about 1300 MPa, about 1400 MPa, about 1500 MPa, about 1600 MPa, about 1700 MPa, about 1800 MPa, about 1900 MPa, about 2000 MPa, about 2100 MPa, about 2200 MPa, about 2300 MPa, about 2400 MPa, about 2500 MPa, about 26500 MPa, about 2700 MPa, about 2800 MPa, about 2900 MPa, about 3000 MPa, about 3500 MPa, or about 4000 MPa.

In any embodiment, the polymer may exhibit elongation at break of 5% to 300%. In any embodiment, the polymer may exhibit improvements in thermal stability of 10% to 70% compared to the generic PLA, which has a melt temperature range of 155 C to 180 C, and thermal decomposition at 220° C. In any embodiment, the polymer may exhibit reduction in boiling shrinkage by 10% to 100%, as generic PLA shrinks substantially (e.g., 40% to 80% shrinkage). In any embodiment, the hydrolytic degradation is improved by 5% to 90%, compared to generic PLA weight/mass loss in hydrolytic settings (acidic, basic, or other aqueous settings similar to wash cycles of textiles).

### Extrusion of Apparel-Grade Filaments

The apparel-grade filament (i.e., the polymer) of the present disclosure is generated using a special combination of elements that act synergistically to produce unexpectedly high quality apparel-grade filament with improved/maintained tensile strength, elongation/flexibility, reduced hydrolysis and boiling shrinkage, improved thermal stability, softer feel of the fabric compared to the commercially available PLA-derived polymer. In particular, the process disclosed herein combines stabilizers epoxy-based chain extenders (epoxy-functionalized compatibilizers), impact modifiers, antioxidants, nucleating agents, anti-hydrolysis agents, elasticity agents (e.g., tensile modulus), and tensile strength agents. For example, the apparel-grade filament can be produced by reacting and/or blending the poly-lactic acid polymer with polyether block amide, TNPP, TiO2, carbodiimides, and epoxy-based chain extenders. In another example, the apparel-grade filament can be produced by reacting and/or blending the poly-lactic acid polymer with polyether block amide, polyamides, TNPP, carbodiimides, and epoxy-based chain extenders.

These additives together create a filament/polymer with improved elongation/flexibility, thermal stability, and reduced boiling shrinkage and hydrolysis compared to its generic PLA counterpart while maintaining or improving tensile strength as well (Table 1). The effect of each additive is not linear, but rather produces emergent properties that allow the maintenance of tensile strength, even though most reagents that improve elongation reduce tensile strength. The novel blends or combinations of additives disclosed herein produced a polymer with synergistic and emergent unique properties as disclosed in Table 1 below. For example, the polymer produced by the methods disclosed herein showed improved tensile strength, elongation/flexibility, reduced hydrolysis and boiling shrinkage, improved thermal stability, softer feel of the fabric. The 112.%7 to about 130.73% elongation at break was surprising an unexpected in light of the 8% elongation at break exhibited by commercially available polymer (Table 1).

In any embodiment, the additive may include a plasticizer, chain extenders, nucleating agents, antioxidants, stabilizers, catalysts, impact modifiers, stabilizers, a thermal resistant additive, a tensile strength modifier, an anti-hydrolysis additive, an antimicrobial, shrink reducer, or combinations of two or more thereof.

In any embodiment, the poly(lactic) acid and the additive may be melt blended or reacted together (i.e. reactive extrusion, and/or separate reactions), such as by cross-linking, stereocomplexation, grafting, bulk polymerization, polycondensation, compatibilization, and/or functionalizing of resin backbones. The reacting together may be conducted by extrusion and optionally catalyzed with one or more processing agents (e.g., p-toluenesulfonic acid (TsOH), tris(nonylphenyl) phosphite (TNPP), glycidyl methacrylate (GMA), dicumyl peroxide (DCP), sorbitols, titanium dioxide, talc, lysine triisocyanate, twice-functionalized organo-clay (TFC), di-n-butyltin oxide (DBTO), phenylene diisocyanate (PDI), methylene diphenyl diisocyanate (MDI), aminopropyl triethoxysilane (APTES), ethylene-butyl acrylate-glycidyl methacrylate (EBG), ethylene-methyl acrylate-glycidyl methacrylate (E-MA-GMA), epoxy-based chain extenders, epoxidized soybean (ESO), maleic anhydride (MA), other chain extenders (CE), lacti-glyceride, and/or 1,2-propanediol isobutyl POSS), pyromellitic dianhydride (PMDA), triglycidyl isocyanurate (TGIC), polycarbodiimide (PCD), biocarbon/biochar; vinylsilane; primary amines; sulfhydryls; dispersing agents including but not limited to: mineral/paraffin oil, essential oil, unsaturated polyamine amides, acidic polyesters; lubricants including but not limited to: polyolefin elastomer, allium extract, merken, thymol and R—(−) carvone oil, β-cyclodextrin-2-nonanone inclusion complex, green tea extract; metallic nanoparticles, or other nucleating agents, stabilizers, antioxidants, chain extenders (CE) or impact modifiers that may contain binary or multi-functional groups (e.g, dianhydride, diamine, diisocyanate) and/or multi-epoxide groups. In any embodiment, the one or more processing agents is tris(nonylphenyl) phosphite (TNPP). In any embodiment, the Nucleating agents may be sorbitols or TiO2. In some embodiments, the processing agent comprises polyamides, polyether block amide, tris(nonylphenyl) phosphite (TNPP), chain extenders (e.g., multi-functional epoxy-based compatibilizer), TiO2, sorbitols, or carbodiimides (epoxy or non-epoxy version).

In any embodiment, the additives provided herein may exhibit one or more properties (e.g., improves tensile strength and shrink reduction properties). In any embodiment, the additives from any category can be used for a different function within the polymer.

### Plasticizers

In any embodiment, the additive may include a plasticizer. In any embodiment, the plasticizer may include polyalkylene glycol (e.g., polyethylene glycol or polypropylene glycol), acetyl tributyl citrate (ATBC), polyalkylene succinate (e.g., polyethylene succinate, polypropylene succinate, or polybutylene succinate), polyethylene oxide, poly(butylene succinate-co-adipate), poly(hydroxybutyrate-valerate), polyacrylate (e.g., poly(methyl methacrylate)), polyphthalate (e.g., polyethylene terephthalate, dibutyl phthalate, polybutylene adipate terephthalate (PBAT), polybutylene terephthalate (PBT), or poly(trimethylene terephthalate)), polycarbonate (e.g., poly(hexamethylene carbonate)), poly(ε-caprolactone) (PCL), polyhydroxyalkanoates (PHA), polyhydroxybutyrate-co-valerate (PHBV), starch (e.g., gelatinized blends or maleated thermoplastic starch (MATPS)), acetyl cellulose (AcC), polypropiolactone (PPL), poly(alkylene adipate) (e.g., poly(ethylene adipate) or poly(butylene adipate)), poly(ethylene suberate) (PESu), poly(ethylene azelate) (PEAz), poly(alkylene sebacate) (e.g., poly(ethylene sebacate) (PESE) or poly(butylene sebacate) (PESE)), poly(ethylene decamethylate) (PEDe), polyurethane, thermoplastic polyurethane (TPU), oligomeric lactic acid (OLA), polyoxymethylene (POM), poly(3-hydroxybutyrate), Poly(vinyl alcohol) (PVA), Poly(glycolic acid) (PGA), poly(styrene-acrylic-co-glycidyl-methacrylate), ethyle-glycidyl methacrylate copolymer, poly(ethylene-n-butylene-acrylate-co-glycydyl-methacrylate), malenized soy bean and/or linseed oil or any other orangic oil, poly(butylene succinate-co-adipate) (PBSA), sunflower oil, terpene D-limonene, epoxidized soybean oil and/or linseed oil, castor oil, palm oil, isosorbide esters, polyether sulfone, or combinations of two or more thereof. In any embodiment, the plasticizer may include poly(ethylene succinate), poly(butylene succinate), poly(butylene adipate terephalate), thermoplastic polyurethane (TPU), polyamides, polyether block amide, polyethylene oxide, poly(butylene succinate-co-adipate), poly(hydroxybutyrate-valerate), poly(styrene-acrylic-co-glycidyl-methacrylate), ethyle-glycidyl methacrylate copolymer, poly(ethylene-n-butylene-acrylate-co-glycydyl-methacrylate), or combinations of two or more thereof. In any embodiment, a plasticizer may also be referred to as a softener.

### Thermal Resistant Additives

In any embodiment, the additive may include a thermal resistant additive. In any embodiment, the thermal resistant additive may include Myrrh extract, lignin, phosphorous-nitrogen-based flame retardant, cellulose nanocrystals, biocarbon, carbon nanocellulose, carbon fibers, zinc oxide, stearates, poly(3-hydroxybutyrate-co-4-hydroxybutyrate), oxidized starch, ammonium polyphosphate, nano clay, silica (i.e., silicon dioxide and its variations such silica nanoparticles), rice husk (e.g., silica from rice husk), Mg—Al layered double hydroxide (LDH) modified with sodium dodecyl sulfate (SDS), gum rosin and its variations, catechin, zinc acetate, polyamides, polyether block amide, carboamides, epoxided-carboamides, epoxy-based chain extendors, sorbitols, polyethylene oxide, poly(butylene succinate-co-adipate), poly(hydroxybutyrate-valerate), poly(styrene-acrylic-co-glycidyl-methacrylate), ethyle-glycidyl methacrylate copolymer, poly(ethylene-n-butylene-acrylate-co-glycydyl-methacrylate), polyamides, polyether block amide, carbodiimides, epoxided-carbodiimides, TNPP, or combinations of two or more thereof. In any embodiment, the thermal resistant additive may include lignin. In any embodiment, the thermal resistant additive may include Mg—Al layered double hydroxide (LDH) modified with sodium dodecyl sulfate (SDS). In any embodiment, the thermal resistant additive may include polyamides, polyether block amide, carbodiimides, epoxided-carbodiimides, poly(styrene-acrylic-co-glycidyl-methacrylate), ethyle-glycidyl methacrylate copolymer, and/or poly(ethylene-n-butylene-acrylate-co-glycydyl-methacrylate) or any combination of the above.

### Tensile Strength Modifiers

In any embodiment, the additive may include a tensile strength modifier/additive. In any embodiment, the tensile strength modifier may include cellulose fiber (e.g., micro and/or nano forms such as cellulose nanocrystals), chitosan, graphene nano platelets, Mg—Al layered double hydroxide (LDH) modified with stearate, glycidyl methacrylate (GMA), poly(trimethylene carbonate) (PTMC), natural rubber, poly(propylene carbonate) (PPC), dicumyl peroxide (DCP), lysine triisocyanate, twice-functionalized organo-clay (TFC), phenylene diisocyanate (PDI), methylene diphenyl diisocyanate (MDI), ethylene-butyl acrylate-glycidyl methacrylate (EBG), ethylene-methyl acrylate-glycidyl methacrylate (E-MA-GMA), poly(styrene-acrylic-co-glycidyl-methacrylate), ethyle-glycidyl methacrylate copolymer, poly(ethylene-n-butylene-acrylate-co-glycydyl-methacrylate), polyamides, polyether block amide, epoxidized soybean (ESO), p-toluenesulfonic acid (TsOH), tris(nonylphenyl) phosphite (TNPP), sorbitols, titanium dioxide, talc, lysine triisocyanate, twice-functionalized organo-clay (TFC), di-n-butyltin oxide (DBTO), methylene diphenyl diisocyanate (MDI), aminopropyl triethoxysilane (APTES), epoxy-based chain extenders, maleic anhydride (MA), lacti-glyceride, and/or 1,2-propanediol isobutyl POSS), pyromellitic dianhydride (PMDA), sorbitols, TiO2, triglycidyl isocyanurate (TGIC), polycarbodiimide (PCD), carbodiimides, epoxided-carbodiimides, maleic anhydride (MA) or other chain extenders, impact modifiers, grafted polymers, or nucleating agents that may contain binary or multi-functional groups, such as dianhydride, diamine, diisocyanate, ethylene methyl acrylate (EMA) and multi-epoxide groups, or combinations of two or more thereof. In any embodiment, the polymer may be reacted or blended with polyether block amide, TNPP, TiO2, epoxy-based chain extenders, carbodiimides, epoxided-carbodiimide, or any combination of agents from above.

In any embodiment, the tensile strength modifier may include fats (e.g., hydrogenated or non-hydrogenated vegetable oils, animal fats), waxes (e.g., microcrystalline wax, bees wax), plasticizers/emulsifiers (e.g., mineral oil, fatty acids, mono- and diglycerides, triacetin, glycerin, acetylated monoglycerides, glycerol monostearate), low and high molecular weight polymers (e.g., polypropylene glycol, polyethylene glycol, polyisobutylene, polyethylene, polyvinyl acetate) and the like, fillers like talc, dicalcium phosphate, calcium carbonate, silica, and combinations thereof.

In any embodiment, the polymer produced by any of the methods disclosed herein may be reacted or blended with polyether block amide, TNPP, TiO2, epoxy-based chain extenders, or any combination of agents or from above. In a preferred embodiment, the polymer produced by any of the methods disclosed herein may be reacted or blended with about 1% to about 25% polyether block amide, about 0.01% to about 1% TNPP, about 0.01% to about 4% TiO2, about 0.01% to 4% epoxided or nonepoxized chain extenders. In that embodiment, the resulting apparel-grade filaments exhibit superior elongation/flexibility, greater shear strength, high thermal stability, improved crystallinity, and maintain high tensile strength when compared to filaments, or PLA known in the art.

### Anti-Hydrolysis Additive

In any embodiment, the additive may include an anti-hydrolysis additive. In any embodiment, the anti-hydrolysis additive may include biosilicate, acid fillers such as fumaric acid-anionic clay composites (e.g., fumaric acid-LDH), silanol treated nano-silica, poly(hydroxybutyrate) (PHB), poly(butylene succinate) (PBS), poly(3-hydroxybutyrate-co-3-hydroxyvalerate) (PHBV), carboamides, poly(styrene-acrylic-co-glycidyl-methacrylate), ethyle-glycidyl methacrylate copolymer, poly(ethylene-n-butylene-acrylate-co-glycydyl-methacrylate), sunflower oil, carboamides, epoxided-carboamides, primary or secondary phosphates, activated fatty acid ester, or combinations of two or more thereof. In any embodiment, the anti-hydrolysis additive may include fumaric acid intercalated with anionic clays (including layered double hydroxides), such as fumaric acid-anionic clay (e.g., fumaric acid-LDH). In any embodiment, the anti-hydrolysis additive may include carboamides.

In any embodiment, the additive may further include collagen, soy fillers, ZnO, thymol, essential oils, propolis extract, citrate esters, sepiolite, lactiglyceride, maleic anhydride, or combinations of two or more thereof.

In any embodiment, the polymer produced by any of the methods disclosed herein may be reacted or blended with polyether block amide, TNPP, TiO2, epoxy-based chain extenders, an anti-hydrolysis additive described herein, and/or an additive selected from the group consisting of collagen, soy fillers, ZnO, thymol, essential oils, propolis extract, citrate esters, sepiolite, lactiglyceride, maleic anhydride, and combinations of two or more thereof.

In some embodiments, the polymer mixture may be molten using one or more UV stabilizers selected from sterically hindered amine, diphenyl cyanoacrylate, hydroxybenzophenone, benzotriazole derivate, hydroxyphenyltriazine, benzophenones, hydroxyphenyl-triazines, or Benzotriazoles. The one or more UV stabilizers may be used at a concentration from about 0.01% to about 10% or about 0.1% to about 3%.

In some embodiments, the method disclosed herein combines stabilizers epoxy-based chain extenders (epoxy-functionalized compatibilizers), impact modifiers, and antioxidants. In some embodiments, the additive may include one or more antioxidants. In that embodiment, the one or more antioxidant may be selected from retinoids, ascorbic acid, tocopherols, polyphenolic compounds epigallocatechin gallate, quercetin, curcumin, resveratrol, or sterically hindered phenols. The one or more antioxidants may be used at a concentration of about 0.01% to about 10% or about 0.1% to about 3%.

In any embodiment, the additive may be added to the polymer mixture and/or during forming of the polymer. In any embodiment, the polymer mixture and/or the polymer may include about 5 wt % to about 30 wt % of the plasticizer, about 1 wt % to about 15 wt % of the thermal resistant additive, about 0.01 wt % to about 15 wt % of the thermal resistant additive, about 1 wt % to about 15 wt % of the tensile strength modifier, about 0.01 wt % to about 15 wt % of the anti-hydrolysis additive, about 0.01 wt % to about 15 wt % of the anti-hydrolysis additive, about 1 wt % to about 10 wt % of the antimicrobial, about 0.01 wt % to about 10 wt % of the antimicrobial or combinations of two or more thereof. In any embodiment, the polymer mixture and/or the polymer may include about 5 wt % to about 20 wt % of the plasticizer, about 1 wt % to about 5 wt % of the thermal resistant additive, about 1 wt % to about 5 wt % of the tensile strength modifier, about 1 wt % to about 30 wt % of the tensile strength modifier, about 1 wt % to about 5 wt % of the anti-hydrolysis additive about 1 wt % to about 5 wt % of the antimicrobial, or combinations of two or more thereof.

### Biodegradable Polymer-Apparel-Grade Filaments

In some embodiments, the polymer is a textile or fabric. In that embodiment, the fabric is woven, non-woven, and/or knit. In some embodiments, the polymer is a filament or fiber used in a multifilament or monofilament yarn.

In some embodiments, the polymer is a fastener, accessory, or combinations of two or more thereof used in textile applications. In any embodiment, the fastener may be in the form of a button, zipper, snap/press studs, buckle, safety pin, cuff links, brooch, hook and eye, frog fastener, toggle fastener, snap hook/clasp, grommet/eyelet, slider, clasp, decorative fabric patch, squeeze buckle, G-hook, or the like. In some embodiments, the polymer is a single use plastic alternative.

In any embodiment, the accessory may be in the form of a handbag/purse, hand fan, parasol/umbrella, wallet, can, jacket, boots/shoes, cravat, tie, hat, bonnet, belt, suspender, gloves/mittens, necklace, bracelet, anklet, earrings, ring, pin, stockings, watch, eyewear, sash, shawl, scarf, lanyard, socks, travel bag (e.g., suitcase, carrying case, backpack, etc.) or the like.

In any embodiment, the fibers and/or filaments may be processed into an interlocking network to form a yarn and/or thread. The textile may then be woven, non-woven or knit using known techniques, including but not limited to, weaving, knitting, crocheting, knotting, tatting, felting, bonding, stitching, and/or braiding the yarn and/or thread. The textile may be in any known form including, but not limited to, apparel textile (e.g., fabric, cloth, material for clothing), faux leather, faux suede, upholstery, carpeting, filters, insulation, geotextiles, window shades, towels, coverings for tables, beds, and other flat surfaces, tents, nets, balloons, kites, sails, parachutes, crafts (sewing, quilting, and/or embroidery), art, and the like. In any embodiment, the polymer may be in the form of a fiber and/or filament suitable for use in an apparel textile yarn and/or thread.

In any embodiment, the fastener, the accessory, the leather, the fiber, and/or the filament or textile may be formed by (but not limited to) extruding, compounding, molding, electrospinning, wetspinning, meltspinning, non-woven processing, drawing, knitting, weaving, spunbonding the polymer mixture. In any embodiment, the polymer mixture may be molten (e.g., using UV stabilizers).

In any embodiment, the fiber and/or the filament may be combined with another material (e.g., fiber or filament that is not poly(lactic) acid) to for use in a yarn and/or thread. Not limiting examples of materials include synthetic (e.g., polyethylene, polyester, nylon, carbon, polypropylene, spandex, rayon, and the like), animal-based (e.g., wool, alpaca, angora, cashmere wool, silk, and the like), plant-based (e.g., bamboo, cotton, flax, hemp, jute, modal, and the like), mineral-based (e.g., copper, gold, steel, fiberglass, and the like), dyes, or combinations of two or more thereof.

In some embodiments, the polymer is a non-woven textile such as for example leather. In that embodiment, the biodegradable polymer produced by the method disclosed herein is melted down or dissolved and converted into leather. In some embodiments, the biodegradable polymer produced by the methods disclosed herein or the biodegradable polymer disclosed herein may be used as a base to produce leather.

### Methods for Producing a Polymer

Another aspect of the present disclosure provides present disclosure methods for producing a polymer comprising poly(lactic) acid, the method including: providing a feedstock; hydrolyzing the feedstock with an enzyme and fermenting the feedstock with a microbe to produce a mixture; isolating lactic acid from the mixture; and converting the lactic acid to poly(lactic) acid; wherein the enzyme comprises cellulase and microbe comprises Rhizopus oryzae. In some embodiments, the feedstock comprises food waste. In any embodiment, the method may further include adding reacting, grafting, and/or cross-linking an additive to the poly(lactic) acid to produce a polymer mixture and/or forming the polymer with the poly(lactic) acid or the polymer mixture. In any embodiment, the polymer may be biodegradable.

In some embodiments, the microbe may include fungi and/or bacteria. In any embodiment, the microbe may include Aspergillus, Pediococcus, Aerococcus, Carnobacterium, Enterococcus, Tetragenococcus, Vagococcus, Leuconostoc, Oenococcus, Weissella, Streptococcus, Lactococcus, Bacillus, Saccharomyces, Lactobacillus, Rhizopus, or combinations of two more thereof. In any embodiment, the microbe may include Streptococcus bovis, Streptococcus thermophiles, Bacillus coagulans, Saccharomyces cerevisiae, Lactobacillus rhamnosus, Lactobacillus manihotivorans, Lactobacillus plantarum, Lactobacillus paracasei, Lactobacillus delbrueckii subsp. Bulgaricus, Lactococcus lactis, Rhizopus oryzae, or combinations of two more thereof. In any embodiment, the microbe may include Bacillus coagulans, Saccharomyces cerevisiae, Streptococcus thermophiles, Lactobacillus rhamnosus, Lactobacillus delbrueckii subsp. Bulgaricus, Lactobacillus plantarum, Rhizopus oryzae, or combinations of two more thereof. In any embodiment, the microbe may include Rhizopus oryzae. In any embodiment, the microbe may include Aspergillus. In any embodiment, the Rhizopus oryzae may include strain NRRL 395, ATCC 52311, GY18, or combinations of two or more thereof. In some embodiments, the microbe may include Rhizopus oryzae (and its various strains and subspecies), any species of Rhizopus genus (e.g. Rhizopus arrhizus, delemar, microspores, oligosporus, nodosus, or nigricans). In any embodiment, the microbe may include Rhizopus oryzae ATCC 52311. In any embodiment, the microbe may include Rhizopus oryzae NRRL 395.

Another aspect of the present disclosure provides a polymer generated by any of the methods disclosed herein. present disclosure. In some embodiments, the polymer comprises poly(lactic) acid and an additive. In that embodiment, the polymer is biodegradable and exhibits a tensile strength of about 30 MPa to about 90 MPa, a Young modulus of about 500 MPa to about 2700 MPa, or a combination thereof. In any embodiment, the poly(lactic) acid may have a molecular weight (MN) of about 90,000 to about 160,000. In any embodiment, the poly(lactic) acid may have a molecular weight (MN) of about 90,000, about 100,000, about 110,000, about 120,000, about 130,000, about 140,000, about 150,000, about 160,000, about 170,000, about 180,000, about 190,000, about 200,000, about 210,000, about 220,000, about 230,000, about 240,000, about 250,000, about 260,000, about 270,000, about 280,000, about 290,000, or about 300,000.

In any embodiment, the polymer may exhibit a tensile strength of about 30 MPa to about 90 MPa. In any embodiment, the polymer may exhibit a tensile strength of about 50 MPa to about 75 MPa. In any embodiment, the polymer may exhibit a tensile strength of about 30, about 40, about 50, about 60, about 70, about 80, about 90, about 100, about 125, about 150, about 175, about 200 MPa. In any embodiment, the polymer may exhibit a Young modulus of about 500 MPa to about 2700 MPa. In any embodiment, the polymer may exhibit a Young modulus of about 500 MPa, about 600 MPa, about 700 MPa, about 800 MPa, about 900 MPa, about 1000 MPa, about 1100 MPa, about 1200 MPa, about 1300 MPa, about 1400 MPa, about 1500 MPa, about 1600 MPa, about 1700 MPa, about 1800 MPa, about 1900 MPa, about 2000 MPa, about 2100 MPa, about 2200 MPa, about 2300 MPa, about 2400 MPa, about 2500 MPa, about 26500 MPa, about 2700 MPa, about 2800 MPa, about 2900 MPa, about 3000 MPa, about 3500 MPa, or about 4000 MPa.

In any embodiment, the polymer may be a fastener, an accessory, fiber, and/or a filament as disclosed herein.

In any embodiment, the additive may be any additive disclosed herein. In any embodiment, the additive may be in the amounts disclosed herein.

In another aspect, the present disclosure provides a method for producing a polymer (e.g., a biodegradable polymer, fiber or filament suitable for use in an apparel textile yarn and/or thread) comprising: providing a lactic acid from a fermented food waste mixture; converting the lactic acid to poly(lactic) acid; adding, reacting, grafting, and/or cross-linking an additive to the poly(lactic) acid to produce a polymer mixture; and forming the polymer with the polymer mixture. In some embodiments, the food waste comprises a high concentration of complex polysaccharides. In some embodiments, the food waste is hydrolyzed with an enzyme disclosed herein and fermented with a microbe disclosed herein.

In yet another aspect, the present disclosure provides a plastic alternative, a single use plastic alternative, a fabric, yarn, cloth or textile comprising the polymer (e.g., biodegradable) described herein and/or a fabric, yarn, cloth or textile made by the method of producing a polymer described herein. In some embodiments, the polymer is about 10%, about 20%, about 30%, about 40%, about 50%, about 60%, about 70%, about 80%, about 90% or about 100% poly-lactic acid polymer. In some embodiments, the fabric, yarn, cloth or textile comprises a non-cotton cellulosic fiber, filament, yarn, textile or fabric.

### Definitions

Unless defined otherwise, all technical and scientific terms used herein have the meaning commonly understood by a person skilled in the art to which this invention belongs.

Technical and scientific terms used herein have the meanings commonly understood by one of ordinary skill in the art, unless otherwise defined. Any suitable materials and/or methodologies known to those of ordinary skill in the art can be utilized in carrying out the methods described herein.

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context.

As used herein, the term “Comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

Recitation of ranges of values herein are merely intended to serve as a shorthand method of referring individually to each separate value falling within the range, unless otherwise indicated herein, and each separate value is incorporated into the specification as if it were individually recited herein.

All numerical designations, e.g., mass, temperature, time, and concentration, including ranges, are approximations which are varied (+) or (−) by increments of 1, 5, or 10%. It is to be understood, although not always explicitly stated that all numerical designations are preceded by the term “about.”

All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, the term “About” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term. In some embodiments, the term “about” indicates the designated value±up to 10%, up to 5%, or up to ±1%.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S¹⁵ are thus within the scope of the present disclosure. Procedures for inserting such labels into the compounds of the present disclosure will be readily apparent to those skilled in the art based on the disclosure herein.

In general, the term “Substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls, sulfonamides; amines; N-oxides; hydrazines; hydrazides; hydrazones; azides; amides; ureas; amidines; guanidines; enamines; imides; isocyanates; isothiocyanates, cyanates; thiocyanates; imines; nitro groups; nitriles (i.e., CN); and the like.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, isobutyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, —C≡CCH₂CH(CH₂CH₃)₂, among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. Although the phrase “Aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Alkoxy groups are hydroxyl groups (—OH) in which the bond to the hydrogen atom is replaced by a bond to a carbon atom of a substituted or unsubstituted alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “Alkanoyl” and “Alkanoyloxy” as used herein can refer, respectively, to —C(O)-alkyl groups and —O—C(O)-alkyl groups, each containing 2-5 carbon atoms. Similarly, “aryloyl” and “aryloyloxy” refer to —C(O)-aryl groups and —O—C(O)-aryl groups.

As used herein, the terms “Aryloxy” and “Arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “Carboxylate” as used herein refers to a —COOH group.

The term “Ester” as used herein refers to —COOR⁷⁰ and —C(O)O-G groups. R⁷⁰ is a substituted or unsubstituted alkyl, cycloalkyl, alkenyl, alkynyl, aryl, aralkyl, heterocyclylalkyl or heterocyclyl group as defined herein. G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, NY, (3rd Edition, 1999) which can be added or removed using the procedures set forth therein and which is hereby incorporated by reference in its entirety and for any and all purposes as if fully set forth herein.

As used herein, the term “Amide” (or “Amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

As used herein, the term “Nitrile” or “cyano” refers to the —CN group.

As used herein, “Urethane groups” include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H. The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

As used herein, the term “Sulfonamido” includes S- and N-sulfonamide groups, i.e., —SO₂NR⁷⁸R⁷⁹ and —NR⁷⁸SO₂R⁷⁹ groups, respectively. R⁷⁸ and R⁷⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

As used herein, the term “Thiol” refers to —SH groups, while “sulfides” include —SR⁸⁰ groups, “sulfoxides” include —S(O)R⁸¹ groups, “sulfones” include —SO₂R⁸² groups, and “sulfonyls” include —SO₂₀R⁸³. R⁸⁰, R⁸¹, R⁸², and R⁸³ are each independently a substituted or unsubstituted alkyl, cycloalkyl, alkenyl, alkynyl, aryl aralkyl, heterocyclyl or heterocyclylalkyl group as defined herein. In some embodiments, the sulfide is an alkylthio group, —S-alkyl.

As used herein, the term “Urea” refers to —NR⁸⁴—C(O)—NR⁸⁵R⁸⁶ groups. R⁸⁴, R⁸⁵, and R⁸⁶ groups are independently hydrogen, or a substituted or unsubstituted alkyl, alkenyl, alkynyl, cycloalkyl, aryl, aralkyl, heterocyclyl, or heterocyclylalkyl group as defined herein.

As used herein, the term “Amidine” refers to —C(NR⁸⁷)NR⁸⁸R⁸⁹ and —NR⁸⁷C(NR⁸⁸)R⁸⁹, where R⁸⁷, R⁸⁸, and R⁸⁹ are each independently hydrogen, or a substituted or unsubstituted alkyl, cycloalkyl, alkenyl, alkynyl, aryl aralkyl, heterocyclyl or heterocyclylalkyl group as defined herein.

As used herein, the term “Guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, alkynyl, aryl aralkyl, heterocyclyl or heterocyclylalkyl group as defined herein.

As used herein, the term “Enamine” refers to —C(R⁹⁴)═C(R⁹⁵)NR⁹⁶R⁹⁷ and —NR⁹⁴C(R⁹⁵)═C(R⁹⁶)R⁹⁷, wherein R⁹⁴, R⁹⁵, R⁹⁶ and R⁹⁷ are each independently hydrogen, a substituted or unsubstituted alkyl, cycloalkyl, alkenyl, alkynyl, aryl aralkyl, heterocyclyl or heterocyclylalkyl group as defined herein.

As used herein, the term “Halogen” or “Halo” refers to bromine, chlorine, fluorine, or iodine. In some embodiments, the halogen is fluorine. In other embodiments, the halogen is chlorine or bromine.

As used herein, the term “Hydroxyl” as used herein can refer to —OH or its ionized form, —O⁻.

As used herein, the term “Hydroxyalkyl” group is a hydroxyl-substituted alkyl group, such as HO—CH₂—.

As used herein, the term “imide” refers to —C(O)NR⁹⁸C(O)R⁹⁹, wherein R⁹⁸ and R⁹⁹ are each independently hydrogen, or a substituted or unsubstituted alkyl, cycloalkyl, alkenyl, alkynyl, aryl aralkyl, heterocyclyl or heterocyclylalkyl group as defined herein.

As used herein, the term “Imine” refers to —CR¹⁰⁰(NR¹⁰¹) and —N(CR¹⁰⁰R¹⁰¹) groups, where R¹⁰⁰ and R¹⁰¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R¹⁰⁰ and R¹⁰¹ are not both simultaneously hydrogen.

As used herein, the term “Nitro” as used herein refers to an —NO₂ group.

As used herein, the term “Trifluoromethyl” as used herein refers to —CF₃.

As used herein, the term “Trifluoromethoxy” as used herein refers to —OCF₃.

As used herein, the term “Azido” refers to —N₃.

As used herein, the term “Trialkyl ammonium” refers to a —N(alkyl)₃ group. A trialkylammonium group is positively charged and thus typically has an associated anion, such as halogen anion.

As used herein, the term “Isocyano” refers to —NC.

As used herein, the term “Isothiocyano” refers to —NCS.

As used herein, the term “Substantially free” refers to less than about 2 wt % of the specified component based on the total weight of the composition. In any embodiment, the composition may include less than about 1 wt %, less than about 0.5 wt %, or less than about 0.1 wt %. In any embodiment, the composition may free of detectable amounts of the component.

As used herein, the term “Biodegradable” refers to the capability of being decomposed by chemical hydrolysis, bacteria, or other living organisms. In any embodiment, the biodegradable polymer disclosed herein may decompose fully in less than about 10 years. In any embodiment, the biodegradable polymer disclosed herein may decompose fully in less than about 9 years, less than about 8 years, less than about 7 years, less than about 6 years, less than about 5 years, less than about 4 years, less than about 3 years, less than about 2 years, or less than about 1 year. In any embodiment, the biodegradable polymer disclosed herein may decompose fully in about 3 months to about 10 years. In any embodiment, the biodegradable polymer disclosed herein may decompose fully in about 3 months to about 5 years. In any embodiment, faster biodegradation occurs if the polymer is under thermophilic conditions (>60° C.), aerobic conditions, and/or aqueous soil medium of 40-60%.

As used herein, the term “Plasticizer additive” refers to an additive that makes a polymer softer and/or more flexible/elongate/elastic. In any embodiment, the plasticizer additive may increase flexibility/elongation by at least about 5%, at least about 10%, at least about 20%, at least about 25%, at least about 50%, at least about 60%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 230%, at least about 250%, at least about 275%, or at least about 300%. In any embodiment, the plasticizer additive may increase flexibility/elongation by at least about 50% to at least about 300%, at least about 50% to at least about 200%, at least about 100% to at least about 250%, at least about 100% to at least about 200%, at least about 150% to at least about 250%, at least about 150% to at least about 200%; or at least about 150% to at least about 300%.

As used herein, the term “Thermal resistance additive” refers to an additive that reduces chemical and/or physical degradation by heating of the polymer. In any embodiment, the thermal resistance additive may inhibit thermal degradation by at least about 5%, at least about 10%, at least about 20%, at least about 25%, at least about 50%, at least about 60%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 230%, at least about 250%, at least about 275%, or at least about 300%.

As used herein, the term “Tensile strength modifier additive” refers to an additive that increases the maximum stress/strain that the polymer can withstand while being stretched or pulled before breaking. In any embodiment, the tensile strength modifier additive may increase the tensile strength of the polymer by at least about 5%, at least about 10%, at least about 20%, at least about 25%, at least about 50%, at least about 60%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 230%, at least about 250%, at least about 275%, or at least about 300%. By employing a tensile strength modifier, the overall tensile strength of the biodegradable polymer can be adjusted or altered in such a way that a preselected tensile strength is obtained for the corresponding desired release profile of the active component from the biodegradable polymer based on a comparison with a standard.

In any embodiment, the tensile strength modifier additive may increase the tensile modulus (i.e., Young's modulus or stiffness) of the polymer by at least about 5%, at least about 10%, at least about 20%, at least about 25%, at least about 50%, at least about 60%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210^{%}, at least about 230%, at least about 250%, at least about 275%, or at least about 300%.

As used herein, the term “Anti-hydrolysis additive” refers to an additive that reduces chemical degradation by the addition of water to the polymer, especially at carbonyl groups of the polymer. The additive may increase hydrolysis stability against acids, bases, and/or moisture (including acidic or alkaline aqueous mediums). In any embodiment, the anti-hydrolysis additive may inhibit hydrolysis by at least about 5%, at least about 10%, at least about 20%, at least about 25%, at least about 50%, at least about 60%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 230%, at least about 250%, at least about 275%, or at least about 300%.

As used herein, the term “Antimicrobial additive” refers to an additive that inhibits microbial growth. Antimicrobial additives/agents may be either at the surface or within the fastener, accessory, fiber, and/or filament. In any embodiment, the antimicrobial additive may inhibit microbial growth by at least about 5%, at least about 10%, at least about 20%, at least about 25%, or at least about 50%.

As used herein, the term “Shrink reducer additive” refers to additives that reduce the effect of heat-based shrinkage especially drier shrinkage and/or hot water shrinkage. In any embodiment, the shrink reducer additive may reduce shrinkage by at least about 5%, at least about 10%, at least about 20%, at least about 25%, or at least about 50%.

As used herein, the term “Food waste” refers to any food-based feedstock. In some embodiments, Food waste is any food biomass that is meant to be thrown away, for recycling, destined for landfill, or compost. The food waste comprises a high concentration of complex saccharides that are hard to hydrolyze. The feedstock may include any part of the food including skin, peels, hulls, husks, bagasse, pomace, seeds, stems, leaves, roots, etc. In some embodiments, food waste includes fruit and/or vegetable biomass, such as any source of pectin such as plant peel and pomace including citrus, orange, grapefruit, potato, tomato, grape, mango, gooseberry, carrot, sugar-beet, and apple, among others. In some embodiments, the food waste comprises agricultural waste byproducts or side streams such as pomace, corn steep liquor, corn steep solids, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, pits, fermentation waste, straw, lumber, sewage, garbage or food leftovers. These materials can come from farms, forestry, industrial sources, households, etc. The food can be processed or unprocessed, homogenous or mixed, dried, frozen, or in any other known form.

As used herein, the term “Feedstock” refers to biomass being used for a process.

As used herein, the term “Complex polysaccharide” is used interchangeably with “Plant polysaccharide.” Plant polysaccharide as used herein has its ordinary meaning as known to those skilled in the art and may comprise one or more carbohydrate polymers of sugars and sugar derivatives as well as derivatives of sugar polymers and/or other polymeric materials that occur in plant matter. Exemplary plant polysaccharides include lignin, cellulose, starch, pectin, and hemicellulose. Others are chitin, sulfonated polysaccharides such as alginic acid, agarose, carrageenan, porphyran, furcelleran and funoran. Generally, the polysaccharide can have two or more sugar units or derivatives of sugar units. The sugar units and/or derivatives of sugar units may repeat in a regular pattern, or otherwise. The sugar units can be hexose units or pentose units, or combinations of these. The derivatives of sugar units can be sugar alcohols, sugar acids, amino sugars, etc. The polysaccharides can be linear, branched, cross-linked, or a mixture thereof. One type or class of polysaccharide can be cross-linked to another type or class of polysaccharide. Plant polysaccharide can be derived from genetically modified plants. Examples of polysaccharides, oligosaccharides, monosaccharides or other sugar components of biomass include, but are not limited to, alginate, agar, carrageenan, fucoidan, pectin, gluronate, mannuronate, mannitol, lyxose, cellulose, hemicellulose, glycerol, xylitol, glucose, mannose, galactose, xylose, xylan, mannan, arabinan, arabinose, glucuronate, galacturonate (including di- and tri-galacturonates), rhamnose, and the like.

As used herein, the term “Saccharification” has its ordinary meaning as known to those skilled in the art and can include conversion of plant polysaccharides to lower molecular weight species that can be used by the microorganism at hand. For some microorganisms, this would include conversion to monosaccharides, disaccharides, trisaccharides, and oligosaccharides of up to about seven monomer units, as well as similar sized chains of sugar derivatives and combinations of sugars and sugar derivatives. For some microorganisms, the allowable chain-length can be longer (e.g. 8, 9, 10, 11, 12, 13, 14, 15, 16, 17, 18, 19, or 20 monomer units or more) and for some microorganisms the allowable chain-length can be shorter (e.g. 1, 2, 3, 4, 5, 6, or 7 monomer units).

### EXAMPLES

The examples herein are provided to illustrate advantages of the present disclosure and to further assist a person of ordinary skill in the art with preparing or using the polymers of the present disclosure. The examples herein are also presented in order to more fully illustrate the preferred aspects of the present disclosure. The examples should in no way be construed as limiting the scope of the present disclosure, as defined by the appended claims. The examples can include or incorporate any of the variations, aspects or aspects of the present disclosure described above. The variations, aspects or aspects described above may also further each include or incorporate the variations of any or all other variations, aspects or aspects of the present disclosure.

### Example 1: Overview

All reagents and materials are or were purchased from commercial vendors.

The present disclosure relates to a circular solution for producing an apparel-grade polylactic acid fabric, a biodegradable synthetic textile alternative produced from post-industrial food waste. The present disclosure is exclusive in using post-industrial food waste, processed through immobilized fungal fermentation technology to create sterically pure L-lactic acid with minimal byproducts, which is then purified and used as input for PLA production. The PLA is then extruded with performance-enhancing additives to form filaments/fibers that are transformed into textiles for apparel applications (but not limited for this). The performance-enhancing additives include (but not limited to): stabilizers (i.e. TNPP), strength elastomers (i.e. polyether block amides), nucleating agents (i.e. titanium dioxide), antihydrolysis agents (i.e. epoxided and non-epoxides carbodiimides), and epoxy-based chain extendors, etc. These additives together create a filament/polymer with improved elongation/flexibility, thermal stability, and reduced boiling shrinkage and hydrolysis compared to its generic PLA counterpart while maintaining or improving tensile strength as well (Table 1). The effect of each additive is not linear, but rather produces emergent properties that allow the maintenance of tensile strength, even though most reagents that improve elongation reduce tensile strength. Previous industrial solutions either have used crops or very specific food by-products to produce biodegradable polymers, however none that create an apparel-grade PLA fabric from a wider variety of food waste sources.

The apparel/fashion industry is projected to account for a quarter of the world's greenhouse gas emissions by 2050. Derived from petroleum, polyester textiles produce 10% of global greenhouse gas emissions annually. There are limited sustainable material solutions, such as recycled polyester, which poses as a temporary solution due to its lack of recyclability and continued contribution to microplastic pollution.

As an alternative to petroleum derived plastics, biodegradable polymers are becoming a popular solution, with examples such as PBS, PHAs, PCL, PLA, etc. In particular, polylactic acid (PLA) is a biobased, biodegradable polymer that belongs to the polyester family. Currently, PLA is increasingly popular for biodegradable straws, cutlery, yard waste bags, drug delivery, and other biomedical applications. Despite the increased market for biodegradable polymers for such applications, biodegradable thermoplastic polymers have been unsuccessful in entering the fashion industry to replace petroleum-based synthetic textiles. This is largely due to the poor durability of biodegradable polymers for apparel applications. Furthermore, biodegradable polymers have relatively lower tensile strength, faster hydrolytic degradation, lower thermal stability, and higher brittleness (lower flexibility) compared to non-biodegradable synthetics, among other performance differences. However, in comparison to other biodegradable polymers, polylactic acid (PLA) is a good candidate for a synthetic textile replacement, if modified and optimized for apparel applications, as it has a similar molecular size to polyester monomers and moderate tensile strength. With its current performance, generic PLA is rendered unsuitable for apparel applications. The durability, tensile strength, thermal stability, elongation/flexibility, and processability (i.e. crystallinity) of PLA must be improved for applicability in the fashion industry.

In terms of the input and production, PLA is derived from lactic acid, industrially generated via bacterial fermentation of renewable feedstocks/crops such as corn, beets, sugar cane, and cassava. The lactic acid then undergoes lactide synthesis and polymerization to form PLA of various molecular weights depending on application.

The present disclosure presents a circular solution that utilizes post-industrial food waste for its input in the production of fabrics for apparel. Post industrial food waste is defined as liquid or solid waste generated during any stage of commercial or industrial food or beverage processes (i.e. wasted crops, cull produce, crop byproducts (e.g. begasse), pomace or juice waste, solid food waste after production of food products). Other PLA producers utilize food crops such as beets, sugar cane, corn/maize, cassava for their polymer production, consuming resources such as land, water, chemicals, and diverting from food supply.

Other sustainable textile producers use materials such as recycled polyester to produce fabric, orange peels to make viscose, mushrooms to make leather and/or food to make other types of biodegradable polymers such as PHAs or PBS which are not equipped as competitive synthetic textile alternatives due to the unmatched mechanical performance and durability of non-biodegradable synthetics like polyester. Furthermore, PBS is currently only partially producible from renewable feedstock while the rest still requires petroleum-based inputs. In comparison to other biodegradable thermoplastic polymers, the present innovation creates a biodegradable PLA-based synthetic textile alternative that is designed for apparel applications, attributed to its optimized tensile strength, tensile modulus, thermal stability, durability, and elongation/flexibility. The polymer made by the method disclosed herein is industrially compostable at the end of its lifecycle, unlike polyester. However, the polymer made by the method disclosed herein is designed to withstand the wash and dry cycles within apparel lifecycle, eventually undergoing microbial degradation once the product is composted.

The present innovation is novel in converting food waste to fabric via production of apparel-grade PLA through fungal fermentation and unique blend of performance-enhancing additives. The present invention is applicable for a wide variety of post-industry food waste that can have various sugar/saccharide composition (i.e. can be cellulose dominant, sucrose dominant, glucose dominant, fructose dominant, etc.). It is not simply dependent on starch-rich sources, which is the main crop type used by other producers. The post-industrial food waste is sterilized and hydrolyzed into glucose or disaccharide molecules depending on the polysaccharide content.

### Food Waste to Lactic Acid

Pre-Fermentation: The food waste can undergo pre-fermentation prep, such as grinding, sterilizing, hydrolysis, etc. It may skip steps such as hydrolysis if the waste input is already contains short saccharides (i.e. monosaccharides, disaccharides). The present innovation avoids the use of acid or basic treatments feedstock/food waste to minimize the use of harsh solvents and mitigate production of harmful byproducts. The present disclosure utilizes enzymes that hydrolyze various types of polysaccharides within the food waste into glucose or disaccharide molecules. The hydrolysis can involve a single enzyme or a blend of enzymes that customized for the food waste type.

Fermentation: One of the core components of ALT TEX technology is the use of immobilized forms of NRRL 395 Rhizopus oryzae. This fungal species is specific for L-Lactic acid and produces minimal levels of byproducts. ALT TEX Rhizopus has been optimized for L-lactic acid yields higher than 135 g/L, whereas the literature states values as high as 100 g/L for semi-continuous or continuous fermentations. The optimized performance is achieved through procedures involving adaptive evolution over 3 years (will continue for longer), genetic modification techniques (i.e. random mutagenesis cycles, shotgun mutagenesis based tools, etc), self-immobilization method/Pellet formation, physical/scaffold immobilization methods (on a scaffold of various shapes and materials). Through these various techniques, the present invention generates enhanced strains of Rhizopus Oryzae NRRL 395 (but not limited to this strain) for L-lactic acid production with optimized conditions. Immobilization of the microbe is a substantial contributing factor in increasing yield from the original 40 g/L (not immobilized) value, whereas the genetic modification and adaptive evolution techniques have reduce byproducts in addition to improving lactic acid yield. The byproducts from our fermentation process are ethanol, fumaric acid, acetic acid, etc. Through the present invention, the fumaric acid production has been minimized to 0.0 g/L, ethanol to less than 0.6 g/L, and acetic acid to less than 0.01 g/L. Another aspect to note is that MgO and MgOH based pH control is uncommon, which is the method utilized by the present disclosure for its eco-friendly procedure and non-toxic byproducts in comparison it's the industry standard of using CaCO3 which creates significant amount of toxic byproduct (calcium sulphate, also known as gypsum). The combination of fungal immobilization and MgO/MgOH₂ has not been done before. The MgO/MgOH₂ also allows the self-immobilized or physically immobilized Rhizopus to be viable in the fermentation, whereas many other pH control agents (aside from CaCO₃) reduce the efficacy of the fungal lactic acid production.

### Lactic Acid to PLLA

In the next phase of the development, the L-lactic is purified via centrifugation, activated carbon use, filtration, rotary evaporation, and ionic exchange chromatography or liquid-liquid extraction (simple or reactive extractions). Ionic exchange chromatography has not been used at scale in the industry. It is mainly liquid extraction mechanisms that are scaled in the industry.

Next, the purified L-lactic acid is converted to Lactide via dehydration, oligomerization, and depolymerization. The present disclosure uses customized temperature, pressure, catalysts, monomer:catalyst ratios, and initiator/co-catalysts systems to optimize yields.

Other PLA producers are creating grades of the polymer that are based on molecular weight differences, and stereo-complexation. While these differences give the polymer versatility in levels of biodegradation and extrusion conditions/methods, it does not cater the uses for apparel functionality. Hence, the present disclosure focuses on fabric properties to back engineer those properties through PLA polymerization (optimizing Mw, reacting antihydrolysis agents and stabilizers during the polymerization process) and using customized combination of additives downstream (extrusion phase).

Depolymerization: During this reaction, the catalyst, monomer:catalyst ratios, co-catalyst/initiator systems are important part of technology. Please refer to list in the patent document.

Ring-opening polymerization: During this reaction, the catalyst, monomer:catalyst ratios, co-catalyst/initiator systems are important part of technology. Please refer to list in the patent document.

### PLLA to Apparel-Grade Filaments

The novel Blends or combinations of additives disclosed herein produced a polymer with synergistic and emergent unique properties as disclosed in Table 1 below. For example, the polymer produced by the methods disclosed herein showed improved tensile strength, elongation/flexibility, reduced hydrolysis and boiling shrinkage, improved thermal stability, softer feel of the fabric. The 112.%7 to about 130.73% elongation at break was surprising an unexpected in light of the 8% elongation at break exhibited by commercially available polymer. Table 1.

### Example 2: Random Mutagenesis of Rhizopus oryzae

Spores are produced by growing Rhizopus oryzae NRRL 395 strain on Potato Dextrose (PD) agar slants at 25-35 C for 7-28 days. Spore suspension is collected with a pipette by rinsing spore mycelia with sterile water. 1-10 mL of spore suspension is exposed to UV irradiation at 254 nm for 1-40 min at 10-25 cm distance. Treated spores are spread on selective agar plates for 48-96 hours at 25 C-35 C; selective agar can contain about 20-80 g/L glucose, 0.1-15 g/L (NH4)2SO4, 0.3 g/L KH2PO4, 0.04 g/L ZnSO4·7H2O, 0.25 g/L MgSO4·7H2O, 1-20 g/L CaCO3, 15 g/L agar, 0.1 g/L bromocerol green and 40-60 mg/L nystatin. Single spore colonies with the largest diameter coloured rings are selected for lactic acid production and inoculated into a 20-60 mL Potato Dextrose medium for 58-96 hours at 25-35 C and agitated at 100-250 rpm. The highest lactic acid yield culture as measured by HPLC using the Aminex 87H column are subjected to a subsequent mutagenesis cycle. The mutagenesis cycle is repeated 3 to 10 times and resulting mutant spores are stored in 25% glycerol stocks at −80 C until further use. [

### Example 3: Coffee Husk Food Waste Converted to Poly(Lactic) Acid

Coffee husk in water (75 g/L) was added into a bioreactor equipped with probes for pH, temperature, and oxygen concentration monitoring. Cultured Rhizopus oryzae (about 5×10⁶ to about 5×10⁷ spores/mL) and 30 mL of cellulase (30 mL) were added to the bioreactor. In some cases, 2 w/w % cellulase by weight percentage with respect to feedstock weight was used. The mixture was stirred at 50 rpm by a mechanical stirrer and warmed to 40° C. Sodium hydroxide was added as needed to maintain a pH of about 6. Following reaction, the product mixture was filtered and centrifuged to provide lactic acid. The lactic acid was treated with activated carbon to remove colored impurities followed by rotary evaporation, sonification, separation, and distillation to provide concentrated L-lactic acid with a 99% purity. In some cases, the lactide was heated to about 200° C. for 25 minutes under nitrogen to produce poly(lactic acid) with 600:1 monomer to catalyst ratio using bismuth subsalicylate or tin octanoate.

The concentrated lactic acid (>99% mL) was heated to 150° C. over 5 hours under nitrogen gas (4000 Pa) to provide oligomeric poly(lactic) acid. Following the addition of zinc oxide catalyst, the oligomeric poly(lactic) acid was depolymerized to lactide under vacuum distillation (195° C. at below 400 Pa). As the yield of lactide began to decrease, sodium bicarbonate was added as a co-catalyst. The lactide was collected in an ice bath and recrystallized at about 4° C. in ethyl acetate. The crystallized precipitate was collected and over dried (40° C. at 100 Pa) for 24 hours to provide lactide.

The lactide was heated to about 200° C. for 25 minutes under nitrogen to produce poly(lactic) acid. The poly(lactic) acid was purified by precipitation in cold methanol and the precipitate was collected and over dried (40° C. at 100 Pa) for 24 hours to provide poly(lactic) acid with a molecular weight of about 75,000 to about 125,000 g/mol (MN).

### Example 4: Fruit Pomace Food Waste Converted to Poly(Lactic) Acid

Following the same method of Example 1, poly(lactic acid with a 99% purity was produced substituting apple pomace for coffee husk.

### Example 5: Potato Peel Food Waste Converted to Poly(Lactic) Acid

Powdered potato peels in water (180 g/L) was heated between 80° C. to 90° C. to undergo gelatinization. The gelatinized potato and water (potato concentration at 60 g/L) were added into a bioreactor equipped with probes for pH, temperature, and oxygen concentration monitoring. Cellulase and β-glucanase (2 wt %) and amylase (2 wt %) were added to the bioreactor. The mixture was stirred at 100 rpm by a mechanical stirrer and warmed to 50° C. to allow hydrolysis. Magnesium oxide was added as needed to maintain a pH of about 6. Following hydrolysis, the cultured Rhizopus oryzae immobilized on polyethylene glycol scaffold was added into the bioreactor for fermentation at 25° C., pH 6, 100 rpm, air 0.75 vvm. The pH was again controlled by the addition of MgO as needed. The product mixture was filtered and centrifuged to provide lactic acid. The lactic acid was treated with activated carbon to remove colored impurities followed by rotary evaporation, acidulation, extraction with alcohol, reverse extraction with water, and distillation to provide concentrated L-lactic acid with a >99% purity.

The concentrated L-lactic acid was heated to 150° C. over 3 hours with pressure decreased from 30,000 Pa to 10,000 Pa to provide oligomeric poly(lactic) acid. In some cases, The concentrated L-lactic acid was heated to 170° C. over 3 hours with pressure decreased from 30,000 Pa to 10,000 Pa to provide oligomeric poly(lactic acid).

Following the addition of zinc oxide catalyst, the oligomeric poly(lactic) acid was depolymerized to lactide under vacuum distillation (195° C., pressure drop from 30,000 Pa to below 400 Pa). In some cases, Zinc-acetate was added to the lactide and heated to about 180° C. for 3 hours under nitrogen to produce poly(lactic acid) with pressure adjustment from 20,000 Pa adjusted down to 2000 Pa. The lactide was collected in an ice bath and recrystallized at about 4° C. in toluene. The crystallized precipitate was collected and oven dried (40° C. at 100 Pa) for 24 hours.

Zinc-acetate was added to the lactide and heated to about 150° C. for 3 hours under nitrogen to produce poly(lactic) acid. The poly(lactic) acid was purified by precipitation in cold methanol and the precipitate was collected and oven dried (40° C. at 100 Pa) for 24 hours to provide poly(lactic) acid with a molecular weight of 70,000 g/mol to 110,000 g/mol (MN).

### Example 6: Polymer Produced with Poly(Lactic) Acid and Additive

The poly(lactic) acid from Example 1 (70-97%) was mixed with thermoplastic polyurethane (TPU) (3-30%) and over dried at 55-80° C. for 4-10 hours to produce a polymer mixture. Ethanol and DBTO (0.1-5 wt %) were added to the polymer mixture followed by twin-screw extrusion (speed 75-2000 rpm) at a temperature of 150-230° C. to produce a polymer.

### Example 7: Polymer Produced with Poly(Lactic) Acid and Additive

This example demonstrates that a polymer produced by the method disclosed herein generated a product that is more elastic and/or softer that commercially available products. As shown in Table 1 below, the elongation at break for commercial PLA is about 8%, whereas the elongation at break of the polymer produced by the method disclosed herein is about 112% to about 130.7% depending on the additive blends or combinations. For example, blend comprising about 0.001% to 10% TNPP (narrow range closer to 0.05 to 0.5%); about 0.001% to 10% epoxy-based chain extenders (narrow range of 0.001% to 2%), about 0.001% to 10% nucleating agents (i.e. TiO2, narrow range 0.01% to 3%); about 0.001% to 10% anti-hydrolysis agents (i.e. carbodiimides, narrow range 0.01% to 2%); and about 1% to 70% elasticity/tensile strength agents (polyether block amides, narrow range of 5% to 25%) generated a polymer comprising synergistic and emergent properties, such as improved tensile strength, elongation/flexibility, reduced hydrolysis and boiling shrinkage, improved thermal stability, and softer feel of the fabric:

TABLE 1

| Blends or Combinations of | Tensile | | Tensile | | Elongation | DSC |
|---|---|---|---|---|---|---|
| additives | Modulus | | Strength | | at break | analysis |
| Commercial PLA (not ALT | 2.6 | GPa | 67 | MPa | 8% | Tc: 57.7 C. |
| TEX PLA | | | | | | Tg: 61 C. |
| *reference values that | | | | | | Tm: 162.4 C. |
| showcase non-apparel grade | | | | | | |
| PLA | | | | | | |
| ALT TEX PLA + epoxy- | 2.69 | GPa | 65.2 | MPa | 122.7% | Tc: 118.4 C. |
| based chain extender + | | | | | | Tg: 50 C. |
| TNPP + polyether block | | | | | | Tm: 173 C. |
| amides (*elongation and | | | | | | |
| tensile strength additive*) | | | | | | |
| ALT TEX PLA + epoxy- | 2.72 | GPa | 69 | MPa | 130.73% | — |
| based chain extender + | | | | | | |
| TNPP + polyether block | | | | | | |
| amides + TiO2 (nucleating | | | | | | |
| agent) | | | | | | |
| ALT TEX PLA + epoxy- | 2.75 | GPa | 66.8 | MPa | 112.7% | — |
| based chain extender + | | | | | | |
| TNPP + polyether block | | | | | | |
| amides + carbodiimides (anti | | | | | | |
| hydrolysis agent) | | | | | | |

### Example 8: Maple Syrup Residue Converted to L-Lactic Acid Via Self-Immobilization Method

This example demonstrates how to produce L-lactic acid via fungal self-immobilization.

Inoculum: 9×125 mL vessels with 20 mL of PD media and 15 g/L CaCO3, following by autoclave/sterilization was prepped. 2.2×10⁷ spores was inoculated into each vessel and incubated for about 20 hours at 30° C. at 200 rpm. The entire content was then poured into a sterilized reactor.

Fermentation: salts and glucose were dissolved in 20 L water in reactor and used 150:1 C:N ratio was used as shown in the Table 2 below. Anti-foam was prepared using 1.5M MgO at 30° C. with stirring, salt solution, and 4 L of 50% glucose and fed-batch salts solution. The reactor was set at a temperature: 30 C, agitation 100 RPM, Air: 0.75 vvm-15 LPM, pH control: S (with 1.5M MgO), DO: Set point of 50%, increase airflow to max 2 vvm, don't adjust agitation. All 125 mL flasks of pellets were inoculated into the reactor. To begin the fermentation, samples were collected after inoculation (Ti) and the fermentation was run for 65-72 hours. 1 L of 50% (w/v) glucose solution (based on sugars dropping) was aspirated from the fermentation and 4 times sampling to total 2000 g of glucose was added over the fermentation.

TABLE 2

| | | Amount for | Amount for fed- |
|---|---|---|---|
| Salt | Ferm g/L | 20 L (g) | batches (g/L) |
| Glucose | 35 | 700 | 500 |
| MgSO4 7H20 | 0.25 | 5 | 0.25 |
| ZnSO4 7H20 | 0.04 | 0.8 | 0.04 |
| (NH4)2SO4 | 0.23 | 4.7 | 3.3 |
| KH2PO4 | 0.3 | 6 | 0.3 |

Harvesting and filtration. All fermentation broth were collected by centrifuging at 4000×g 30 min if required. Activated carbon granules were then mixed in and sieved. The solution was then filtered through 0.45 um filter TFF. Concentrate the solution (e.g., using rotovap) to a desired concentration of L-lactic acid (e.g., e.g. 80 g/L lactic acid).

### Example 9: L-Lactic Acid (from Fermentation Broth) to PLA

Dehydration: A 250 mL two-necked RBF equipped with a Dean-Stark trap and an air condenser was set on a hot plate. Commercial lactic acid (50 mL, >86%) was added to a round-bottom flask (250 mL) at using the conditions disclosed in Table 3 in order to remove water content. After completion, a sample is taken and analyzed by 1H-NMR in in deuterated chloroform.

TABLE 3

| Time | Temperature | Pressure |
|---|---|---|
| (min) | (° C.) | (kPa) |
| 0 | 60 | 15 |
| 20 | 60 | 10 |
| 30 | 65 | 10 |
| 40 | 65 | 7.5 |
| 50 | 70 | 7.5 |
| 60 | 70 | 6 |
| 70 | 75 | 6 |
| 80 | 80 | 6 |
| 90 | 80 | 6 |
| 110 | 80 | 6 |
| Time x (until no | 85 | 6 |
| water collected) | | |

Oligomerization: After dehydration, conditions can be adjusted to oligomerization settings as highlighted in table 3. After the theoretical water volume is collected (estimated according to the scheme shown below), turn off the heat and vacuum and cool the reaction mixture to room temperature.

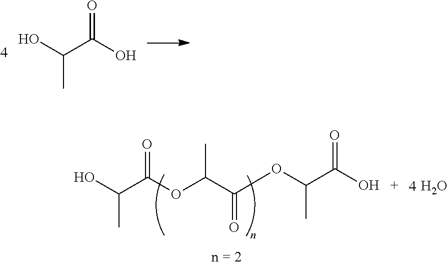

An oligomer sample is taken and analyzed by ¹H-NMR spectroscopy in deuterated methanol and by GPC to determine the molecular weight distribution

Depolymerization: Continuing from Step 2 setup, turn off the N₂ and set up the distillation apparatus (see picture below). Distillation apparatus is wrapped with fiber glass. Next, adjust the conditions to depolymerization settings as highlighted in the same table, at timepoint of catalyst addition. Pressure is lowered to allow simultaneous lactide isolation from the reaction system by distillation until no more product came out (Table 4). After the reaction is stopped by turning the heat and vacuum off, water and lactic acid oligomer were obtained in the collecting Schlenk flask. From the 2-necked RBF, the lactide formed (white crystals).

TABLE 4

| Time | Temperature | Pressure | Instructions/ |
|---|---|---|---|
| (min) | (° C.) | (kPa) | notes |
| 0 | 80 | 15 | |
| 10 | 90 | 15 | |
| 20 | 100 | 15 | |
| 30 | 110 | 15 | |
| 40 | 120 | 15 | |
| 50 | 130 | 15 | |
| 60 | 140 | 15 | |
| 90 | 150 | 15 | |
| 120 | 150 | 10 | |
| 130 | 160 | 10 | |
| 140 | 160 | 7.5 | |
| 150 | 170 | 7.5 | |
| 160 | 170 | 5 | |
| 170 | 180 | 5 | |
| 180 | 180 | 3 | |
| 190 | 185 | 3 | |
| 200 | 185 | 1 | |

Switch to depolymerization

| | | | |
|---|---|---|---|
| 240 | 185 | 10 | Add 0.6% ZnO or |
| | | | creatinine or any |
| | | | other catalyst |
| 250 | 185 | 5 | |
| 260 | 190 | 5 | |
| 270 | 190 | 3 | |
| 280 | 200 | 3 | |
| 290 | 200 | 1 | |
| 300 | 210 | vac | |

Recrystallization using toluene. The crude product is transferred to 1-necked RBF (250 mL) and solubilized with 100 mL of toluene at 80° C. After cooling down, the RBF was transferred into a flammable freezer (<−10° C.) to crystallize the product over 24 h. After crystallization, the crystals were filtered using a fritted funnel and dried under vacuum (50 mTorr) for 24 h at 40° C.

Ring opening polymerization can be conducted using the following steps. Dry a three-neck round-bottom flask under oven and pre-heat an oil bath to 110° C. Evacuate the round bottom flask to create vacuum, and then purge the flask with nitrogen. Charge L-lactide (10 g, 139 mmol, 200 eq) and into a round-bottom flask. Dry the lactide under vacuum. While purging the system with nitrogen, add 2 mL of toluene (0.2 mL/g) to the reaction flask. Insert the flask into the oil bath. Insert the overhead stirrer into the reaction flask, without stirring. When the lactide is fully dissolved/melted, insert the stirrer blade into the lactide and start stirring. Dissolve desired amount of tin octanoate in toluene (0.1 mL/g of lactide), or dissolve desired amount of tin octanoate in 0.5 mL of toluene and dodecanol (or another initiator) in 0.5 mL of toluene. Ensure oil bath is at the desired temperature, then add catalyst/initiator to reaction flask under nitrogen once lactide has fully melted/dissolved

The polymerization was carried out in a pre-heated oil bath at 110° C. under a nitrogen flow. Retrieve sample for monitoring by ¹H NMR (CDCl₃) and GPC (THF) as needed. Stop the reaction when the lactide conversion has reached completion or has plateaued. Completion can be determined by assessing the sample. First, the sample would likely harden as it is removed from the reactor. Collect sample using a spatula. If using a pipette, sample needs to be scraped off from the outside of the pipette. Second, sample may also require sonication to be dissolved in CDCl₃. For dissolving in THF, heating may be required. The flask is cooled down to room temperature. Use a spatula to break up the polymers as it cools to prevent the polymer from hardening as one piece.

Rapid Purification: The crude PLA product is mixed with chloroform:methanol solution of 1:4 ratio at 60° C. for 30 minutes and then filtered. Total PLA to solvent ratio is approximately 1:2 w:v. Lastly, the purified PLA will be dried under the Schlenk line under vacuum at 60° C. for 4 h. A sample can be taken and analyzed by ¹H-NMR in CDCl₃ and GPC in THF or chloroform.

### Example 10: Apparel-Grade Polymer Produced with Poly(Lactic Acid) and Additive

Conditions: 0.11% TNPP+1% epoxy-based chain extenders+1% nucleating agent (i.e. TiO2)+1.5% anti-hydrolysis agents (i.e. carbodiimides)+10% elasticity/tensile strength agents (polyether block amides) reacted or blended together create synergistic and emergent effects (improved tensile strength, elongation/flexibility, reduced hydrolysis and boiling shrinkage, improved thermal stability), softer feel of the fabric, etc.

Parameters: PLA and polymer were put into twin-screw extruder alongside a catalyst. Ensuring that materials are recorded as described above. Extruder parameters are as follows and recorded a functional temperature range of about 170-220° C.; with recommended starting conditions disclosed in Table 5. The Screw speed of the extruder may be set at a functional range of 20-110 rpm, with a recommended start at 100 rpm.

TABLE 5

| In-feed temp. (° C.) | Melt Profile (° C.) | Die Temp (° C.) |
|---|---|---|
| 45-60 | 180-190-195-190 | 190 |

### EQUIVALENTS

While certain embodiments have been illustrated and described, a person with ordinary skill in the art, after reading the foregoing specification, can effect changes, substitutions of equivalents and other types of alterations to the compounds of the present disclosure or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present disclosure is also not to be limited in terms of the particular aspects described herein, which are intended as single illustrations of individual aspects of the present disclosure. Many modifications and variations of this present disclosure can be made without departing from its spirit and scope, as will be apparent to those skilled in the art. Functionally equivalent methods within the scope of the present disclosure, in addition to those enumerated herein, will be apparent to those skilled in the art from the foregoing descriptions. Such modifications and variations are intended to fall within the scope of the appended claims. It is to be understood that this present disclosure is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present disclosure indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

### INCORPORATION BY REFERENCE

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

1.-117. (canceled)

118. A method for producing a polymer, the method comprising:
(a) providing a feedstock, wherein the feedstock comprises food waste;
(b) hydrolyzing the feedstock with an enzyme and fermenting the feedstock with a microbe to produce a mixture;
(c) isolating lactic acid from the mixture;
(d) converting the lactic acid to poly(lactic) acid;
(e) adding, reacting, grafting, and/or cross-linking an additive to the poly(lactic) acid to produce a polymer mixture; and
(f) forming the polymer with the polymer mixture, wherein the polymer is biodegradable.

119. A method of producing a biodegradable polymer comprising poly(lactic acid), the method comprising:
(a) providing a lactic acid;
(b) converting the lactic acid to oligo(lactic acid);
(c) depolymerizing the oligo(lactic acid) to produce cyclic lactide with a catalyst selected from the group consisting of Zn(La)₂, sodium bicarbonate, Sn(OEt)₂, ZnO, TnO, Zn acetate, or combinations of two or more thereof;
(d) polymerizing the cyclic lactide to produce the poly(lactic acid); and
(e) contacting the poly(lactic acid) with an additive to produce a polymer mixture.

120. A method for producing a polymer comprising poly(lactic) acid, the method comprising:
(a) providing a feedstock, wherein the feedstock comprises food waste;
(b) hydrolyzing the feedstock with an enzyme and fermenting the feedstock with a microbe to produce a mixture;
(c) isolating lactic acid from the mixture;
(d) converting the lactic acid to poly(lactic) acid;
(e) adding, reacting, grafting, and/or cross-linking an additive to the poly(lactic) acid to produce a polymer mixture; and/or
(f) forming the polymer with the poly(lactic) acid or the polymer mixture; and
(g) optionally, extruding or pelletizing the polymer into multifilament fibers, wherein the enzyme comprises cellulase and microbe comprises Rhizopus oryzae; and wherein the polymer is biodegradable.

121. The method of claim 118, wherein the additive comprises a processing agent, a plasticizer, a thermal resistant additive, a tensile strength modifier, an impact modifiers, an anti-hydrolysis additive, an antimicrobial, shrink reducer, or combinations of two or more thereof.

122. The method of claim 121, wherein:
(a) the plasticizer comprises polyalkylene glycol, acetyl tributyl citrate (ATBC), polyalkylene succinate, polyethylene oxide, poly(butylene succinate-co-adipate), poly(hydroxybutyrate-valerate), polyacrylate, polyphthalate, polycarbonate, poly(ε-caprolactone) (PCL), polyhydroxyalkanoates (PHA), polyhydroxybutyrate-co-valerate (PHBV), starch, acetyl cellulose (AcC), polypropiolactone (PPL), poly(alkylene adipate), poly(ethylene suberate) (PESu), poly(ethylene azelate) (PEAz), poly(alkylene sebacate), poly(ethylene decamethylate) (PEDe), polyurethane, thermoplastic polyurethane (TPU), oligomeric lactic acid (OLA), polyoxymethylene (POM), poly(3-hydroxybutyrate), Poly(vinyl alcohol) (PVA), Poly(glycolic acid) (PGA), terpene D-limonene, epoxidized soybean oil and/or linseed oil, castor oil, palm oil, isosorbide esters, polyether sulfone, or combinations of two or more thereof; or
(b) the processing agent comprises polyamides, polyether block amide, tris(nonylphenyl) phosphite (TNPP), chain extenders, optionally a multi-functional epoxy-based compatibilizer, TiO2, sorbitols, or carbodiimides, optionally an epoxy or a non-epoxy version; or
(c) the thermal resistant additive comprises Myrrh extract, lignin, phosphorous-nitrogen-based flame retardant, poly(3-hydroxybutyrate-co-4-hydroxybutyrate), oxidized starch, ammonium polyphosphate, nano clay, silica, rice husk, Mg—Al layered double hydroxide (LDH) modified with sodium dodecyl sulfate (SDS), gum rosin and its variations, catechin, zinc acetate, or combinations of two or more thereof, or
(d) the tensile strength modifier comprises cellulose fiber, chitosan, graphene nano platelets, Mg—Al layered double hydroxide (LDH) modified with stearate, glycidyl methacrylate (GMA), poly(trimethylene carbonate) (PTMC), natural rubber, poly(propylene carbonate) (PPC), dicumyl peroxide (DCP), lysine triisocyanate, twice-functionalized organo-clay (TFC), phenylene diisocyanate (PDI), methylene diphenyl diisocyanate (MDI), ethylene-butyl acrylate-glycidyl methacrylate (EBG), ethylene-methyl acrylate-glycidyl methacrylate (E-MA-GMA), epoxidized soybean (ESO), maleic anhydride (MA), chain extenders that may contain binary or multi-functional groups selected from dianhydride, diamine, diisocyanate, and multi-epoxide groups, or combinations of two or more thereof, or
(e) the anti-hydrolysis additive comprises biosilicate, fumaric acid-anionic clay composite, silanol treated nano-silica, poly(hydroxybutyrate) (PHB), poly(butylene succinate) (PBS), poly(3-hydroxybutyrate-co-3-hydroxyvalerate) (PHBV), or combinations of two or more thereof; and/or
(f) the additive further comprise collagen, soy fillers, ZnO, thymol, essential oils, propolis extract, citrate esters, sepiolite, lactiglyceride, maleic anhydride, or combinations of two or more thereof.

123. The method of claim 121, wherein the polymer mixture comprises:
(a) about 5 wt % to about 30 wt % of the plasticizer, about 1 wt % to about 15 wt % of the thermal resistant additive, about 1 wt % to about 15 wt % of the tensile strength modifier, about 1 wt % to about 15 wt % of the anti-hydrolysis additive, about 1 wt % to about 10 wt % of the antimicrobial, or combinations of two or more thereof, or
(b) about 5 wt % to about 20 wt % of the plasticizer, about 1 wt % to about 5 wt % of the thermal resistant additive, about 1 wt % to about 5 wt % of the tensile strength modifier, about 1 wt % to about 5 wt % of the anti-hydrolysis additive about 1 wt % to about 5 wt % of the antimicrobial, or combinations of two or more thereof.

124. The method of claim 118, wherein the polymer is:
(a) extruded and/or formed into:
(i) a plastic alternative, or a single use plastic alternative; and/or
(ii) a fastener, an accessory, a fiber, a yarn, a textile, a fabric, or a filament suitable for use in a textile yarn and/or thread, or combinations of two or more thereof,
optionally wherein the polymer mixture is molten,
optionally wherein the fiber, the filament, the yarn, the textile or the fabric comprises a non-cotton cellulosic fiber, filament, yarn, textile or fabric; or
(b) a fiber or filament suitable for use in an apparel textile yarn and/or thread; or
(c) dope-dyed prior to the extrusion step.

125. The method of claim 118, wherein:
(a) the food waste comprises starch-based food waste, fructose-based food waste, cellulose-based food waste, sucrose-based food waste, or combinations of two or more thereof; or
(b) the food waste comprises coffee husk, potato peel, or a combination thereof.

126. The method of claim 118, wherein:
(a) the enzyme comprises cellulase, amylase, glucoamylase, beta-glucanase, beta-glucosidase, pectinase, hemicellulase, xylanase, arabanase, pectinase, or combinations of two or more thereof,
(b) the microbe comprises fungi and/or bacteria;
(c) the microbe comprises Aspergillus, Pediococcus, Aerococcus, Carnobacterium, Enterococcus, Tetragenococcus, Vagococcus, Leuconostoc, Oenococcus, Weissella, Streptococcus, Lactococcus, Bacillus, Saccharomyces, Lactobacillus, Rhizopus, or combinations of two more thereof,
(d) the microbe comprises Streptococcus bovis, Streptococcus thermophiles, Bacillus coagulans, Saccharomyces cerevisiae, Streptococcus thermophiles, Lactobacillus rhamnosus, Lactobacillus manihotivorans, Lactobacillus plantarum, Lactobacillus paracasei, Lactobacillus delbrueckii subsp. Bulgaricus, Lactococcus lactis, Rhizopus oryzae, or combinations of two more thereof,
(e) the microbe comprises a Rhizopus oryzae strain selected from NRRL 395 or a variant thereof, ATCC 52311, GY18, or combinations of two or more thereof,
(f) the microbe comprise Rhizopus oryzae NRRL 395;
(g) the microbe is immobilized; or
(h) the microbe is genetically modified.

127. The method of claim 118, wherein:
(a) a weight ratio of the enzyme to the microbe is about 5:1 to about 1:5, or about 2:1 to about 1:2;
(b) the mixture comprises about 0.1 wt % to about 10 wt % of the enzyme; or about 1 wt % to about 3 wt % of the enzyme;
(c) the lactic acid comprises L-lactic acid;
(d) the lactic acid comprises at least about 50 wt % of the L-lactic acid, optionally wherein the L-lactic acid comprises about 75 wt % to about 100 wt % of the L-lactic acid; or
(e) the lactic acid is substantially free of D-lactic acid; or
(f) the method does not include an acid and/or alkali treatment of the food waste.

128. The method of claim 118, wherein:
(a) wherein the hydrolyzing and/or the fermenting is conducted at a temperature between about 10° C. and about 75° C., about 40° C. and about 60° C., or about 15° C. and about 35° C.; and/or at a pH between about 4 and about 8 or between about 5.5 and about 7.5.
(b) the isolating the lactic acid comprises filtering, concentrating, extracting, distilling, adding activated carbon and filtering, sonification, or combinations of two or more thereof;
(c) the converting the lactic acid to the poly(lactic) acid comprises:
(i) an azeotropic dehydrative condensation process,
(ii) a direct polycondensation process, or
(iii) a ring opening polymerization process, optionally wherein the ring opening polymerization process comprises converting the lactic acid to oligo(lactic acid), depolymerizing the oligo(lactic acid) to produce cyclic lactide, and polymerizing the cyclic lactide to produce the poly(lactic) acid;
(d) the adding the poly(lactic) acid and the additive comprises melt blending the poly(lactic) acid and the additive; or
(e) the poly(lactic) acid has a molecular weight of about 90,000 to about 160,000.

129. The method of claim 128, wherein:
(a) the converting the lactic acid to the oligo(lactic acid) comprises heating, or removing water;
(b) the depolymerizing the oligo(lactic acid) to produce cyclic lactide comprises distilling, heating, or adding a catalyst, optionally wherein the catalyst is selected from Zn(La)₂, sodium bicarbonate, Sn(OEt)₂, ZnO, TnO, Zn acetate, or combinations of two or more thereof,
(c) the polymerizing the cyclic lactide to produce the poly(lactic) acid comprises heating; or
(d) the cyclic lactide is crystallized one or more times prior to the polymerizing.

130. The method of claim 118, further comprising:
(a) pre-treating the feedstock in a solvent at a temperature above about 60° C. optionally wherein the solvent comprises water having a pH of about 6.5 to about 7.5, optionally wherein the temperature is about 70° C. to about 110° C., optionally wherein the pre-treating occurs for about 30 minutes to about 90 minutes;
(b) adding a pH adjusting agent following the hydrolyzing and the fermenting, optionally wherein the pH adjusting agent comprises a carbonate, a sodium hydroxide, a hydroxide, an amine, or combinations of two or more thereof, or
(c) purifying the poly(lactic) acid.

131. A biodegradable polymer comprising a poly(lactic acid) made by the method of claim 118.

132. The biodegradable polymer of claim 131, wherein the biodegradable polymer exhibits a tensile strength of about 30 MPa to about 90 MPa, a Young modulus of about 500 MPa to about 2700 MPa, or a combination thereof.

133. A method of producing a lactic acid, the method comprising:
(a) providing a feedstock comprising food waste selected from the group consisting of starch-based food waste, fructose-based food waste, cellulose-based food waste, sucrose-based food waste, or combinations of two or more thereof,
(b) contacting the feedstock with an enzyme blend comprising one or more enzymes;
(c) fermenting the feedstock with an immobilized fungal strain to produce a fermentation mixture; and
(d) isolating lactic acid from the fermentation mixture;
wherein the contacting step comprises grinding, sterilizing, and/or hydrolyzing the feedstock.

134. The method of claim 133, further comprising:
(a) adding a pH adjusting agent to the fermentation mixture during fermentation and/or
(b) pre-treating the feedstock in a solvent at a temperature above about 60° C.

135. The method of claim 133, wherein:
(a) the immobilized fungal strain is added to a composition comprising the feedstock and the one or more enzymes;
(b) the one or more enzymes are added to a composition comprising the feedstock and the immobilized fungal strain;
(c) the pH adjusting agent comprises a carbonate, hydroxide, amines, or combinations of two or more thereof,
(d) the pH adjusting agent comprises sodium hydroxide (NaOH), magnesium hydroxide Mg(OH)₂, magnesium oxide MgO and/or calcium carbonate (CaCO₃); or
(e) the pre-treating occurs for about 30 minutes to about 90 minutes.

136. The method of claim 133, wherein the one or more enzymes comprise cellulase.

137. The method of claim 133, wherein the microbe comprises Rhizopus oryzae, and wherein the Rhizopus oryzae comprises:
(a) a strain selected from the group consisting of NRRL 395, ATCC 52311, GY18, a variant thereof, and/or combinations of two or more thereof, or
(b) Rhizopus oryzae NRRL 395 or a variant thereof.
